(19) United States
(12) Patent Application Publication
(10) Pub. No.: US 2022/0257725 A1
(43) Pub. Date: Aug. 18, 2022

(54) COMPOSITIONS AND METHODS FOR TREATING PEROXISOMAL BIOGENESIS DISORDERS

(71) Applicant: University of Utah Research Foundation, Salt Lake City, UT (US)

(72) Inventors: Esther Nuebel, Salt Lake, UT (US); Jared Rutter, Salt Lake, UT (US); Yu-Chan Chen, San Jose, CA (US); Joshua Bonkowsky, Salt Lake City, UT (US)

(21) Appl. No.: 17/625,514

(22) PCT Filed: Jul. 10, 2020
(86) PCT No.: PCT/US2020/041627

§ 371 Date: Jan. 7, 2022

Related U.S. Application Data
(60) Provisional application No. 62/873,119, filed on Jul. 11, 2019.

(51) Int. Cl. A61K 38/46; A61K 31/197; A61K 31/4015; A61P 3/00

(52) U.S. Cl. CPC A61K 38/46; A61P 3/00; A61K 31/4015; A61K 31/197

(57) ABSTRACT

Disclosed herein are compositions and methods for treating mitochondrial dysfunction. The methods can include compositions capable of increasing or overexpressing ATAD1. The methods include restoring mitochondrial respiration and ATP production. The present disclosure provides a mechanism for peroxin (peroxisomal biogenesis factors) accumulation on mitochondria that affects respiration and ATP generation.

### CROSS REFERENCE TO RELATED APPLICATIONS

This application claims the benefit of U.S. Provisional Applications No. 62/873,119, filed Jul. 11, 2019. The content of this earlier filed application is hereby incorporated by reference herein in their entirety.

### STATEMENT REGARDING FEDERALLY FUNDED RESEARCH

This invention was made with government support under grant number GM115174 awarded by the National Institutes of Health. The government has certain rights in the invention.

### INCORPORATION OF THE SEQUENCE LISTING

The present application contains a sequence listing that is submitted via EFS-Web concurrent with the filing of this application, containing the file name “21101_0400P1_SL.txt” which is 8,192 bytes in size, created on Jul. 6, 2020, and is herein incorporated by reference in its entirety.

### BACKGROUND

Peroxisomal Biogenesis Disorders (PBD; a rare group of metabolic disorders) are caused by inherited mutations of a peroxin genes (e.g., Pex1, Pex12, Pex10, etc.), characterized by mitochondrial dysfunction. PBD encompass a mitochondrial phenotype that affects metabolism and causes multi-organ failure, as well as neurological impairment. Currently, only symptomatic treatment is available including palliative care in the end stage. The available treatments do not reduce the symptoms itself, but are limited to reducing symptom consequences. Strategies to alleviate mitochondrial dysfunction are needed.

### SUMMARY

The present disclosure provides a mechanism for peroxin (peroxisomal biogenesis factors) accumulation on mitochondria that affects respiration and ATP generation. As described herein, overexpressing ATAD1 removes peroxins from the mitochondrial membrane and thus can restore mitochondrial function. As described herein, overexpression of ATAD1 can be useful in treating neurodegenerative diseases and peroxisomal biogenesis disorders.

Described herein are methods of restoring mitochondrial respiration and ATP production.

Described herein are compositions capable of increasing or overexpressing ATAD1.

Disclosed herein are compositions comprising a peptide, wherein the peptide comprises the sequence of SEQ ID NO: 1, or a variant a fragment thereof.

Disclosed herein are pharmaceutical compositions comprising a peptide, wherein the peptide comprises the sequence of SEQ ID NO: 1, or a variant or a fragment thereof and a pharmaceutically acceptable carrier.

Disclosed herein are compositions comprising a nucleic acid capable of encoding a peptide comprising the sequence of SEQ ID NO: 1, or a variant or a fragment thereof.

Disclosed herein are methods of removing one or more peroxins embedded in the outer mitochondrial membrane of a cell in a subject, the method comprising: administering a therapeutically effective amount of any of the compositions disclosed herein.

Disclosed herein are methods of reducing peroxin accumulation in the outer mitochondrial membrane of a cell in a subject, the method comprising: administering a therapeutically effective amount of any of the compositions disclosed herein.

Disclosed herein are methods of rescuing mitochondrial function in a cell of a subject, the method comprising: administering a therapeutically effective amount of any of the compositions disclosed herein.

Disclosed herein are methods of treating a subject that has or has been diagnosed with a perioxisomal biogenesis disorder, the method comprising: administering a therapeutically effective amount of any of the compositions disclosed herein, wherein the therapeutically effective amount of any of the compositions disclosed herein removes one or more peroxins embedded in the outer mitochondrial membrane of a cell in the subject, reduces peroxin accumulation in the outer mitochondrial membrane of a cell in the subject, or rescues mitochondrial function in a cell of the subject, thereby treating the subject.

Disclosed herein are methods of reducing one or more symptoms of a peroxisomal biogenesis disorder in a subject, the method comprising: administering a therapeutically effective amount of any of the compositions disclosed herein, wherein the therapeutically effective amount of any of the compositions disclosed herein removes one or more peroxins embedded in the outer mitochondrial membrane of a cell in the subject, reduces peroxin accumulation in the outer mitochondrial membrane of a cell in the subject, or rescues mitochondrial function in a cell of the subject, thereby reducing one or more symptoms of a perioxisomal biogenesis disorder in the subject.

Disclosed herein are methods of treating a subject with a deficiency in human ATAD1 levels or expression, the method comprising: administering a therapeutically effective amount of any of the compositions disclosed herein, wherein the therapeutically effective amount of any of the compositions disclosed herein removes one or more peroxins embedded in the outer mitochondrial membrane of a cell in the subject, reduces peroxin accumulation in the outer mitochondrial membrane of a cell in the subject, or rescues mitochondrial function in a cell of the subject, thereby treating the subject.

Disclosed herein are methods restoring mitochondrial respiration in a subject, the method comprising: administering a therapeutically effective amount of any of the compositions disclosed herein, wherein the therapeutically effective amount of any of the compositions disclosed herein restores mitochondrial respiration in the subject.

Disclosed herein are methods of restoring mitochondrial respiration in a cell, the method comprising: contacting a cell with a therapeutically effective amount of any of the compositions disclosed herein, wherein the therapeutically effective amount of any of the compositions disclosed herein removes one or more peroxins embedded in the outer mitochondrial membrane of a cell in a subject, reduces peroxin accumulation in the outer mitochondrial membrane of a cell in a subject, or rescues mitochondrial function in a cell of a subject.

Disclosed herein are methods of treating a deficiency in human ATAD1 levels or expression in a cell, the method comprising: contacting a human cell with a therapeutically effective amount any of the compositions disclosed herein, wherein the therapeutically effective amount of any of the compositions disclosed herein removes one or more peroxins embedded in the outer mitochondrial membrane of a cell in the subject, reduces peroxin accumulation in the outer mitochondrial membrane of a cell in the subject, or rescues mitochondrial function in a cell of the subject.

Disclosed herein are methods of increasing human ATAD1 protein or gene levels or expression in a cell in a subject, the method comprising: contacting a cell with a therapeutically effective amount of any of the compositions disclosed herein wherein the therapeutically effective amount of any of the compositions disclosed herein removes one or more peroxins embedded in the outer mitochondrial membrane of a cell in the subject, reduces peroxin accumulation in the outer mitochondrial membrane of a cell in the subject, or rescues mitochondrial function in the cell in the subject.

Disclosed herein are methods inhibiting the import of one or more peroxisomal matrix proteins into the mitochondria of a cell, the method comprising: contacting the cell with a therapeutically effective amount of any of the compositions disclosed herein, wherein the peptide of any of the compositions disclosed herein is capable of binding to the peroxisomal matrix protein.

### BRIEF DESCRIPTION OF THE DRAWINGS

FIGS. 1A-H show that peroxins accumulate on mitochondria in the absence of peroxisomes. FIG. 1A shows yeast cells of each strain (wild-type, msp1Δ, pex19Δ, pex19Δmsp1Δ, pex3Δ and pex19Δmsp1Δ) expressing RFP-SKL (peroxisomal marker) construct and mitochondria-targeted green fluorescent protein (GFP) were grown to mid-log phase and analyzed by fluorescence microscopy. Representative images are shown. DIC, differential interference contrast. Enhanced: the red signal intensity was set to ‘best'fit’ in ZEN microscopy software analysis. FIG. 1B shows yeast cells of each strain (wild-type, msp1Δ, pex19Δ, pex19Δmsp1Δ, pex3Δ and pex19Δmsp1Δ) grown to mid-log phase, back diluted to 1 OD and serial dilutions were dropped onto agar plates containing synthetic media with dextrose (S-D), glycerol (S-Gly), glycerol and oleate (S-Gly-Ole) and oleate (S-Ole). FIG. 1C shows wild-type versus pex19Δ RNA levels blotted. Outliers are indicated. Red dots represent peroxin-RNAs. FIG. 1D shows the fold-change of RNA levels (log 2) versus the cumulative fraction of wild-type versus pex19Δ blotted. The red graph represents the peroxin distribution. FIG. 1E shows the translational efficiency (TE) of wild-type versus pex19Δ. Outliers are indicated. Red dots represent translational efficiency of peroxin genes. FIG. 1F shows pex19Δ versus pex19Δmsp1Δ RNA levels blotted. Outliers are indicated. Red dots represent peroxin-RNAs. FIG. 1G shows fold-change of RNA levels (log 2) versus the cumulative fraction of pex19Δ versus pex19Δmsp1Δ blotted. The red graph represents the peroxin distribution. FIG. 1H shows the translational efficiency (TE) of pex19Δ versus pex19Δmsp1Δ. Outliers are indicated. Red dots represent translational efficiency of peroxin genes.

FIGS. 2A-G show that peroxins accumulate on mitochondria in the absence of peroxisomes. FIG. 2A shows yeast cells of each strain expressing the PEX13-RFP construct and mitochondria-targeted green fluorescent protein (GFP) grown to mid-log phase and analyzed by fluorescence microscopy. Representative images are shown. DIC, differential interference contrast. FIG. 2B shows yeast cells of each strain expressing the PEX11-RFP construct and mitochondria-targeted green fluorescent protein (GFP) grown to mid-log phase and analyzed by fluorescence microscopy. Representative images are shown. DIC, differential interference contrast. FIG. 2C shows a depiction of the sample generation for quantitative mass spectrometry (experimental flow). FIG. 2D shows a volcano plot (log 10) representing the average of 5 biological replicates of each strain (pex19Δ and pex19Δmsp1Δ) to indicate most enriched/decreased proteins in the mitochondrial proteome of pex19Δ and pex19Δmsp1Δ. Detected by quantitative mass spectrometry. FIG. 2E shows a heatmap (log 2) representing 5 biological replicates of each strain (pex19Δ and pex19Δmsp1Δ) and their peroxin protein levels detected by quantitative mass spectrometry, protein classes are indicated. FIG. 2F shows total yeast cell lysate, post mitochondrial soup and nycodenz purified mitochondria of each strain (pex19Δ and pex19Δmsp1Δ; pex3Δ pex3Δmsp1Δ) transformed with Pex13^{V5} (expressed by its endogenous promotor) were separated by SDS-PAGE and immunoblotted for Pex13-V5 (a-V5) and porin (a-porin), cytochrome c (cyt c) and HSP70 (hsp70). FIG. 2G shows total yeast cell lysate, post mitochondrial soup and nycodenz purified mitochondria of each strain (pex19 and pex19msp1; pex3 pex3msp1) transformed with Pex11^{V5} (expressed by its endogenous promotor) were separated by SDS-PAGE and immunoblotted for Pex11-V5 (a-V5) and porin (a-porin), cytochrome c (cyt c) and HSP70 (hsp70).

FIGS. 3A-H show peroxins accumulate in specific assemblies on mitochondria. FIG. 3A shows a depiction of detected peroxins which are discussed to contribute to the peroxisomal importomer on peroxisomes. FIG. 3B shows fractionated yeast cells (total=T, post mitochondrial soup=PMS, mitochondria=mito) expressing PEX13^{V5} (endogenous promotor) from yeast strains (wildtype, pex19Δ, pex19Δmsp1Δ) separated by SDS-PAGE and immunoblotted for PEX13^{V5} (V5) and Porin (porin). FIG. 3C shows digitonin solubilized (1 and 4 g/g detergent/membrane ratio as indicated) mitochondrial membranes expressing PEX13^{V5} (endogenous promotor) from yeast strains (wildtype, pex19Δ, pex19Δmsp1Δ) separated by BN-PAGE with a 3-18% gradient stained with Coomassie brilliant blue. FIG. 3D shows digitonin solubilized (1 and 4 g/g detergent/membrane ratio as indicated) mitochondrial membranes expressing PEX13^{V5} (endogenous promotor) from yeast strains (wildtype, pex19Δ, pex19Δmsp1Δ) separated by BN-PAGE with a 3-18% gradient. Gel was transferred to PVDF membrane and immunoblotted for PEX13^{V5} (V5). FIG. 3E shows the relative abundance as heatmap of indicated peroxins plotted against the apparent molecular mass in BNE gels. Left panel represents the protein abundance in the wild-type, middle panel in pex19Δ and the right panel in pex19Δmsp1Δ. FIG. 3F shows a depiction of detected peroxins which contribute to the peroxisomal importomer assembled on mitochondria. FIG. 3G shows total yeast cell lysate, post mitochondrial soup and nycodenz purified mitochondria of each strain (pex19Δ and pex19Δmsp1Δ and pex13Δpex19Δmsp1Δ) transformed with Pex22^{V5} (expressed by its endogenous promotor) separated by SDS-PAGE and immunoblotted for Pex22 (a-V5) and porin (a-porin). FIG. 3H shows total yeast cell lysate, post mitochondrial soup and nycodenz purified mitochondria of each strain (pex19Δ and pex19Δmsp1Δ and pex13Δpex19Δmsp1Δ) transformed with Mdh3^{V5} (expressed by its endogenous promotor) separated by SDS-PAGE and immunoblotted for Mdh3 (a-V5) and porin (a-porin).

FIGS. 4A-B show PEX13-GFP accumulates on mitochondria in PBD pex3− cell lines. FIG. 4A shows a Western blot of human cell lysates separated on a 10% SDS-gel, transferred to nitrocellulose and decorated with antibodies to detect ATAD1-HA-flag expression with a-HA, PEX3-V5 expression with a-V5, GFP-control and PEX13-GFP expression with a-GFP and VDAC as loading control with a-VDAC. FIG. 4B shows fluorescence microscopy of patient fibroblast cell lines: cWT (wild-type), cWT+ATAD1 (wild-type expressing ATAD1) PEX3− (patient), PEX3−+ATAD1 (PEX3− expressing ATAD1) expressing PEX13-GFP stained with Mitotracker far red (MT) to visualize the mitochondrial network and GFP fused to PEX13 (PEX13-GFP) to investigate the localization.

FIGS. 5A-H shows that ATAD1 rescues the mitochondrial phenotype in PBD pex3 cell lines. FIG. 5A shows electron microscopy of patient fibroblast cell lines: cWT (wild-type), cWT+ATAD1 (wild-type expressing ATAD1) PEX3− (patient), PEX3−+ATAD1 (PEX3− expressing ATAD1). Representative images of the most observed mitochondrial morphology are shown. FIG. 5B shows a bioenergetic profile of human patient fibroblasts. OCR (pmol/min/norm.unit) for cWT (wild-type), cWT+ATAD1 (wild-type expressing ATAD1) PEX3− (patient), PEX3−+ATAD1 (PEX3− expressing ATAD1), cWT-ATAD1 (wildtype with ATAD1 deletion), PEX3−ATAD1 (patient with ATAD1 deletion) cells plotted against time (XF96e Extracellular Flux Analyzer, Mito-Stress-Test.) Oligomycin A (1 μM), 0.15 μM FCCP and 1 μM rotenone+1 μM antimycin A (final concentrations) were sequentially delivered to the XF96e assay medium through injection ports in the sensor cartridge. n=2 (4+4=8 technical replicates). FIG. 5C shows a bar graph of basal respiration measured in (B) cWT (wild-type), cWT+ATAD1 (wild-type expressing ATAD1) PEX3− (patient), PEX3−+ATAD1 (PEX3− expressing ATAD1), cWT-ATAD1 (wildtype with ATAD1 deletion), PEX3−ATAD1 (patient with ATAD1 deletion). Statistical significance (Welch's test), p values are indicated as: ns (not significant), * p≤0.05, ** p≤0.01, *** p≤0.001, **** p≤0.0001. FIG. 5D shows a bar graph of uncoupled respiration measured in (B) cWT (wild-type), cWT+ATAD1 (wild-type expressing ATAD1) PEX3− (patient), PEX3−+ATAD1 (PEX3− expressing ATAD1), cWT-ATAD1 (wildtype with ATAD1 deletion), PEX3−ATAD1 (patient with ATAD1 deletion). Statistical significance (Welch's test), p values are indicated as: ns (not significant), * p≤0.05, ** p≤0.01, *** p≤0.001, **** p≤0.0001. FIG. 5E show a bar graph representation of calculated respiratory spare capacity (=uncoupled respiration minus basal respiration) of cWT (wild-type), cWT+ATAD1 (wild-type expressing ATAD1) PEX3− (patient), PEX3−+ATAD1 (PEX3− expressing ATAD1), cWT-ATAD1 (wildtype with ATAD1 deletion), PEX3−ATAD1 (patient with ATAD1 deletion). Statistical significance (Welch's test), p values are indicated as: ns (not significant), * p≤0.05, ** p≤0.01, *** p≤0.001, **** p≤0.0001. FIG. 5F shows a Box-Whisker-Violin blot representation of the average normalized peak intensity of the detected cardiolipin species, log 10 pareto-scaled. FIG. 5G shows Box-Whisker-Violin blot representation of the average normalized peak intensity of the detected phosphoethanolamin (PE) species, log 10 pareto-scaled. FIG. 5H shows a Box-Whisker-Violin blot representation of the average normalized peak intensity of all detected ether-phospholipid (plasmalogen) species, log 10 pareto-scaled.

FIG. 6 shows the interaction of yeast Pex15 (traditional substrate) and Pex13 (untraditional substrate) with yeast Msp1 trap-mutant. Three % input and ½ of the total eluate fraction from total yeast cell lysate of msp1Δ pex19Δ transformed with Pex15^{GFP} (expressed by its endogenous promotor) (left side) and Pex13^{V5} (expressed by its endogenous promotor) (right side) and either EV (empty vector), native MSP1 or trap-mutant (MSP1-E193Q-HA) (indicated with + for transformed and − not transformed) were separated by SDS-PAGE and immunoblotted for Pex15 with a-GFP and Pex13 with a-V5.

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

As used in the specification and in the claims, the term “comprising” can include the aspects “consisting of” and “consisting essentially of” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds; reference to “a pharmaceutical carrier” includes mixtures of two or more such carriers, and the like.

The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a condition, disease or disorder. The term “patient: includes human and veterinary subjects. In some aspects of the disclosed methods, the “patient” has been diagnosed with a need for treatment for a Peroxisomal Biogenesis Disorder, such as, for example, prior to the administering step. In some aspects, of the disclosed methods, the “patient” has been diagnosed with a need for treatment for Zellweger syndrome, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level.

“Modulate”, “modulating” and “modulation” as used herein mean a change in activity or function or number. The change may be an increase or a decrease, an enhancement or an inhibition of the activity, function or number.

As used herein, the terms “restore mitochondrial function” or “restoring mitochondrial function” refer to the mitochondrial morphology (e.g., removing peroxins embedded in the outer mitochondrial membrane). For example, the mitochondria of the wild type (representing a healthy subject or a subject without a peroxisomal biogenesis disease) displays moderate electron density and invaginations called cristae. A subject, for example, with a peroxisomal biogenesis disorder displays more electron density and almost no visible cristae. In some aspects, a “rescued” mitochondria shows less electron density and more cristae than a mitochondria in a subject with a peroxisomal biogenesis disorder.

As used herein, the terms “restore mitochondrial respiration” or “restoring mitochondrial respiration” refer to the level of mitochondrial energy production (e.g., the amount of mitochondrial basal respiration; amount of uncoupled mitochondrial respiration; and respiratory spare capacity of the mitochondria). For example, any of restoring mitochondrial basal respiration, restoring the amount of mitochondrial uncoupled respiration and restoring the respiratory spare capacity of the mitochondrial can be restored back to the level of mitochondrial basal respiration, mitochondrial uncoupled respiration or respiratory spare capacity of the mitochondrial in a wildtype cell or a cell from or within a subject that does not have a peroxisomal biogenesis disorder. It is known in the art that if basal mitochondrial respiration and respiratory spare capacity of the mitochondrial increase that mitochondria substantially contribute to the overall ATP levels of the cell.

As used herein, the terms, “reduce” or “reducing” mean to diminish or decrease in activity, response, condition, disease, amount, or other biological parameter. In some aspects, this may include a 10% reduction in the activity, response, condition, amount, or disease as compared to the native or control level. Thus, in some aspects, the reduction can be a 10, 20, 30, 40, 50, 60, 70, 80, 90, 100%, or any amount of reduction in between as compared to native or control levels. In some aspects, the reduction can be 10-20, 20-30, 30-40, 40-50, 50-60, 60-70, 70-80, 80-90, or 90-100% as compared to native or control levels. In some aspects, the reduction is 0-25, 25-50, 50-75, or 75-100% as compared to native or control levels. In some aspects, reduce can mean make better. For instance, reduce can mean to improve a sign or a symptom of a disease, disorder or condition or to make a sign or symptom of a disease, disorder or condition less severe.

As used herein the phrase “rescuing mitochondrial function” refers to the improvement of mitochondrial function based on one or more of mitochondrial morphology, basal respiration, uncoupled respiration and respiratory spare capacity.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

As used herein, the term “prevent” or “preventing” refers to preventing in whole or in part, or ameliorating or controlling.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to Peroxisomal Biogenesis Disorder or Zellweger syndrome.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, or affection.

The phrase “nucleic acid” as used herein refers to a naturally occurring or synthetic oligonucleotide or polynucleotide, whether DNA or RNA or a DNA-RNA hybrid, single-stranded or double-stranded, sense or antisense, which is capable of hybridization to a complementary nucleic acid by Watson-Crick base-pairing. Nucleic acids as disclosed herein can also include nucleotide analogs (e.g., BrdU), and non-phosphodiester internucleoside linkages (e.g., peptide nucleic acid or thiodiester linkages). In particular, nucleic acids can include, without limitation, DNA, RNA, cDNA, gDNA, ssDNA, dsDNA or any combination thereof.

As used herein, the term “polypeptide” refers to any peptide, oligopeptide, polypeptide, gene product, expression product, or protein. A polypeptide is comprised of consecutive amino acids. The term “polypeptide” encompasses naturally occurring or synthetic molecules. As used herein, the term “amino acid sequence” refers to a list of abbreviations, letters, characters or words representing amino acid residues.

As used herein, the term “gene” refers to a region of DNA encoding a functional RNA or protein. “Functional RNA” refers to an RNA molecule that is not translated into a protein. Generally, the gene symbol is indicated by using italicized styling while the protein symbol is indicated by using non-italicized styling.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

As used herein, the term “complementary” refers to the ability of a nucleic acid to form hydrogen bond(s) with another nucleic acid sequence by either traditional Watson-Crick or other non-traditional types. A percent complementary indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary).

As used herein, the term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element or regulatory element). The terms “plasmid” and “vector” can be used interchangeably, as a plasmid is a commonly used form of vector. Moreover, this disclosure is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s) such as leaves, roots or meristem. The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

The term “fragment” can refer to a portion (e.g., at least 5, 10, 25, 50, 100, 125, 150, 200, 250, 300, 350, 400 or 500, etc. amino acids or nucleic acids) of a peptide that is substantially identical to a reference peptide and retains the biological activity of the reference. In some aspects, the fragment or portion retains at least 50%, 75%, 80%, 85%, 90%, 95% or 99% of the biological activity of the reference peptide described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, lie, and Val; Glu and Asp; Lys and Arg; Phe, Tyr, and Trp; and Gin, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH2)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

Peroxisomes are present in most eukaryotes and are surrounded by a single membrane. Their conserved functions in fatty acid metabolism and scavenging of reactive oxygen species are shared with the mitochondria (Smith and Aitchison, 2013). The mammalian peroxisome is involved in the biosynthesis of bile acids, purine, and specific lipids, including plasmalogens, a precursor of myelin forming a sheath around neurons. The majority of the peroxisomal reactions occur in the matrix of the organelle, into which more than 50 enzymes are imported. Peroxisomal membrane proteins (PMPs) comprise primarily metabolite transporters and peroxins (Theodoulou et al., 2013). Peroxins are peroxisome biogenesis factors that participate in different aspects of peroxisomal biogenesis, including protein import, inheritance, and division. Given the multitude of metabolic pathways peroxisomes are involved in, a defect in peroxisome function or biogenesis can have a severe phenotype, but little is known about the mechanisms involved.

Peroxisome biogenesis disorders (PBDs) are inherited metabolic and neuropathic diseases that affect a multitude of metabolic pathways, often leading to death in early childhood. Mutations in most genes encoding peroxins are linked to PBDs with various degrees of severity (Delille et al., 2006). PBDs include Zellweger Syndrome (ZSD), neonatal adrenoleukodystrophy and infantile Refsum disease. ZSD has the most severe presentation of the PDBs, with myriad clinical features including: neonatal seizures, hepatomegaly, renal cysts, skeletal abnormalities, impaired hearing, and symptoms related to mitochondrial abnormalities such as weakness, poor eyesight and shortness of breath (Crane, 2014, Wagener et al., 2018). The mitochondrial aspect of the disease in particular is poorly understood. Therefore, described herein are results investigating why peroxisomal disease affects mitochondria and vice versa.

Since there is evidence that several neurological disorders may improve upon correction of mitochondrial defects, exploiting mitochondrial protein quality control as a means to rescue mitochondrial function was also assessed.

Yeast Msp1 and its mammalian homologue ATAD1, which belong to the AAA+ATPase protein family, was shown to facilitate the extraction and degradation of mislocalized tail-anchored proteins from mitochondria (Chen et al., 2014). Msp1 was crystallized and the cryo-EM of the trap-mutant of Msp1 (E193Q) confirmed that Msp1's mode of action is similar to that of p97 and spastin in that it forms a homohexamer with a central pore through which it threads substrate proteins. Unlike p97 and spastin, Msp1/ATAD1 specializes in extracting its substrates from membranes (Wohlever et al., 2017). Targeting this quality control mechanism could be beneficial in the context of mitochondrial protein accumulation and disassembly of complexes (Piard et al., 2018).

Peroxisomes and mitochondria are functionally and physically engaged (Schrader and Yoon, 2007). Mitochondria share and complement peroxisomal metabolic pathways of lipid metabolism and reactive oxygen species defense and may contribute to peroxisomal biogenesis. Recently, a pathway was proposed in which peroxins traffic to the mitochondria and into mitochondrial derived vesicles, which fuse with ER-derived preperoxisomes and give rise to functional peroxisomes (McBride 2017). Investigation of the interorganellar communication between peroxisomes and mitochondria may lead to alternative therapeutic targets and benefit PBD/ZSD patients as well as patients with neurodegenerative diseases such as Alzheimer's disease, Parkinson's disease, multiple sclerosis, and age-related diseases.

Zellweger syndrome (ZSD) is a rare, congenital disorder characterized by the reduction or absence of functional peroxisomes caused by mutations in genes that encode a group of proteins known as peroxins. For example, in Zellweger syndrome, accumulation of very long chain fatty acids (VLCFA) and branched chain fatty acids (BCFA) are found as well as deficient levels of plasmalogens and ether-phospholipids. The signs and symptoms of (ZSD) include but are not limited to impaired neuronal migration, neuronal positioning, and brain development; reduction in central nervous system (CNS) myelin (particularly cerebral); sensorineuronal degeneration leading to progressive hearing and vision loss; and mitochondrial morphology changes.

The neuronal disease spectrum of Zellweger syndrome mainly affects peroxisomal biochemistry by impairment of lipid metabolism. To date, little is known about the interaction between peroxisomes and mitochondria to maintain metabolic homeostasis and their dependence on each other based on their shared metabolic pathways. Patients with Zellweger syndrome present with encephalomyopathies and neurological symptoms, mainly caused by a lack of myelination, which also affects the brain white matter. Interestingly, they also present with mitochondrial dysfunction. Mitochondria were traditionally limited to the view that they provide the cell's energy by respiration. Recently, this view has been challenged by evidence that mitochondria are central to cell signaling as well as in response to stress occurring in metabolic disease. Therefore, this mitochondrial phenotype and the underlying mechanisms in cell lines from ZSD patients was characterized. The Examples disclosed herein show that when overexpressing the ATPase family AAA domain-containing protein 1, ATAD1, a protein acting in mitochondrial quality control, the mitochondrial phenotype is rescued in the peroxisomal compromised cells. Further, the Examples described herein show that mitochondrial stress response mechanisms might have an important clinical impact in the treatment of Zellweger syndrome patients.

Also disclosed herein is a mechanism that causes mitochondrial dysfunction in Zellweger syndrome. Peroxisomal membrane proteins accumulate on mitochondria and assemble into complexes with their peroxisome-native binding partners. Overexpression of ATAD1/Msp1 reduces peroxin accumulation on mitochondria and rescues mitochondrial function in fibroblasts from human patients with Zellweger Syndrome and in pex3Δ yeast. It is thought that the severity of the neurological symptoms in ZSD might improve upon restoring mitochondrial function. The results described herein sheds light on the interorganellar communication between peroxisomes and mitochondria and highlights the possibility to improve PBDs but also other diseases which display protein accumulation in the outer mitochondrial membrane. Improving mitochondrial quality control offers a therapeutic strategy in PBD and may serve as a therapeutic strategy in other neurodegenerative diseases.

Further disclosed herein is the biochemical mechanism that leads to peroxins accumulating in the outer mitochondrial membrane in the absence or malfunction of peroxisomes. This causes a respiration defect and affects mitochondrial amino acid metabolism. The overexpression of ATAD1, a member of the mitochondria quality control system, can result in the removal of the accumulating peroxins and therefore the restoration of respiration and amino acid metabolism. In other words, respiration can be restored, thus, mitochondrial energy production (ATP) is restored. Without ATP, a multitude of processes in the cell cannot be performed and therefore can lead to cell death and metabolic crisis. Targeting this biochemical mechanism may reduce or prevent the consequences of ATP depletion and metabolic crisis and lead to the reduction of neuronal impairment. Also, disclosed herein, are methods of treating a PBD phenotype and compositions comprising a vector that can be used to modulate the expression of ATAD1 (e.g., to increase or overexpress ATAD1). In some aspects, compositions comprising the protein ATAD1 or variants or fragments thereof can be used to modulate the expression of ATAD1 (e.g., to increase or overexpress ATAD1.

Compositions

Disclosed herein are compositions, including pharmaceutical compositions, capable of removing one or more peroxins embedded in the outer mitochondrial membrane of a cell in a subject. Also, disclosed herein are compositions capable of reducing peroxin accumulation in the outer mitochondrial membrane of a cell in a subject. Further, disclosed herein are compositions capable of rescuing mitochondrial function in fibroblasts of a subject. Also, disclosed herein are compositions capable of reducing one or more symptoms of a perioxisomal biogenesis disorder in a subject. In some aspects, the subject has a perioxisomal biogenesis disorder. In some aspects, the perioxisomal biogenesis disorder can be Zellweger Syndrome, neonatal adrenoleukodystrophy, or infantile Refsum disease. In some aspects, the subject has Alzheimer's disease, Parkinson's disease, multiple sclerosis, or an age-related disease.

Disclosed herein are peptides or polypeptides that comprise or consist of the amino acid sequence of: MVHAEAFSRPLSRNEVVGLIFRLTIFGAVTYFTIKWMVDAIDPTRKQKVEAQKQAEK LMKQIGVKNVKLSEYEMSIAAHLVDPLNMHVTWSDIAGLDDVITDLKDTVILPIKKK HLFENSRLLQPPKGVLLYGPPGCGKTLIAKATAKEAGCRFINLQPSTLTDKWYGESQ KLAAAVFSLAIKLQPSIIFIDEIDSFLRNRSSSDHEATAMMKAQFMSLWDGLDTDHSC QVIVMGATNRPQDLDSAIMRRMPTRFHINQPALKQREAILKLILKNENVDRHVDLLE VAQETDGFSGSDLKEMCRDAALLCVREYVNSTSEESHDEDEIRPVQQQDLHRAIEK MKKSKDAAFQNVLTHVCLD (SEQ ID NO: 1; >sp|Q8NBU5|ATAD1_HUMAN ATPase family AAA domain-containing protein 1 OS═Homo sapiens OX=9606 GN=ATAD1 PE=1 SV=1). The amino acid residues in bold can interact with one or more peroxins. In some aspects, the amino acids residues at positions W166, Y167 and/or H206 of SEQ ID NO: 1 can recognize, bind or interact with one or more peroxins. The residues R201 and D205 of SEQ ID NO: 1 also display a growth phenotype in this study, indicative of their participation in substrate recognition (Wang et al. eLife 2020; 9:e54031; Wang et al. is incorporated by reference herein in its entirety). In some aspects, the peptides, polypeptides, fragments or variants disclosed herein can comprise amino acids at positions W166, Y167, R201, D205, H206 of SEQ ID NO: 1 or a combination thereof.

In some aspects, the one or more peroxins can be a tail-anchored protein. In some aspects, the one or more peroxins are not a tail-anchored protein.

Disclosed herein are peptides or polypeptides that comprise or consist of the amino acid sequence of: MVHAEAFSRPLSRNEVVGLIFRLTIFGAVTYFTIKWMVDAIDPTRKQKVEAQKQAEK LMKQIGVKNVKLSEYEMSIAAHLVDPLNMHVTWSDIAGLDDVITDLKDTVILPIKKK HLFENSRLLQPPKGVLLYGPPGCGKTLIAKATAKEAGCRFINLQPSTLTDKWYGESQ KLAAAVFSLAIKLQPSIIFIDEIDSFLRNRSSSDHEATAMMKAQFMSLWDGLDTDHSC QVIVMGATNRPQDLDSAIMRRMPTRFHINQPALKQREAILKLILKNENVDRHVDLLE VAQETDGFSGSDLKEMCRDAALLCVREYVNSTSEESHDEDEIRPVQQQDLHRAIEK MKKSKDAAFQNVLTHVCLD (SEQ ID NO: 2; >sp|Q9D5T0|ATAD1_MOUSE ATPase family AAA domain-containing protein 1 OS=Mus musculus OX=10090 GN=Atad1 PE=1 SV=1). The amino acid residues in bold can interact with one or more peroxins. In some aspects, the amino acids residues at positions W166, Y167 and/or H206 of SEQ ID NO: 2 can recognize, bind or interact with one or more peroxins. Residues R201 and D205 also display a growth phenotype in this study, indicative of their participation in substrate recognition (Wang et al. eLife 2020; 9:e54031; Wang et al. is incorporated by reference herein in its entirety). In some aspects, the peptides, polypeptides, fragments or variants disclosed herein can comprise amino acids at positions W166, Y167, R201, D205, H206 of SEQ ID NO: 2 or a combination thereof.

In some aspects, the one or more peroxins can be a tail-anchored protein. In some aspects, the one or more peroxins are not a tail-anchored protein.

Disclosed herein are peptides or polypeptides that comprise or consist of the amino acid sequence of SEQ ID NO: 1, or a fragment thereof. Disclosed herein are peptides or polypeptides that comprise or consist of the amino acid sequence of SEQ ID NO: 2, or a fragment thereof. In some aspects, the fragment of SEQ ID NO: 1 or SEQ ID NO: 2 can consist or comprise the amino acids residues at positions W166, Y167, R201, D205, H206 or a combination thereof. Disclosed herein are peptides or polypeptides that comprise or consist of the amino acid sequence of SEQ ID NO: 1, or a variant thereof. Disclosed herein are peptides or polypeptides that comprise or consist of the amino acid sequence of SEQ ID NO: 2, or a variant thereof. In some aspects, the variant of SEQ ID NO: 1 or SEQ ID NO: 2 can consist or comprise the amino acids residues at positions W166, Y167, R201, D205, H206 or a combination thereof.

In some aspects, the peptides, polypeptides, fragments or variants thereof described herein can bind to or interact with one or more peroxins. In some aspects, the one or more peroxins can be a tail-anchored protein. In some aspects, the one or more peroxins are not a tail-anchored protein.

Disclosed herein are peptides or polypeptides that comprises a fragment of SEQ ID NO: 1. In some aspects, the fragments thereof can comprise a sequence having at least 20%, 30%, 40%, 50%, 60%, 70%, 80%, 90%, 95%, 98%, 99% identity to SEQ ID NO: 1. In some aspects, the fragment of SEQ ID NO: 1 has an amino acid sequence of at least 90% or 95% sequence identity to SEQ ID NO: 1. In some aspects, the fragment retains at least 50%, 75%, 80%, 85%, 90%, 95% or 99% of the biological activity of the reference protein described herein.

Disclosed herein are peptides or polypeptides that comprises a variant of SEQ ID NO: 1. In some aspects, the variants can comprise a sequence having at least 20%, 30%, 40%, 50%, 60%, 70%, 80%, 90%, 95%, 98%, 99% identity to SEQ ID NO: 1. In some aspects, the variant of SEQ ID NO: 1 has an amino acid sequence of at least 90% or 95% sequence identity when compared to SEQ ID NO: 1. In some aspects, the variants retains at least 50%, 75%, 80%, 85%, 90%, 95% or 99% of the biological activity of the reference protein described herein.

Disclosed herein are peptides or polypeptides that comprises a fragment of SEQ ID NO: 2. In some aspects, the fragments thereof comprises a sequence having at least 20%, 30%, 40%, 50%, 60%, 70%, 80%, 90%, 95%, 98%, 99% identity to SEQ ID NO: 2. In some aspects, the fragment of SEQ ID NO: 2 has an amino acid sequence of at least 90% or 95% sequence identity to SEQ ID NO: 2. In some aspects, the fragment retains at least 50%, 75%, 80%, 85%, 90%, 95% or 99% of the biological activity of the reference protein described herein.

Disclosed herein are peptides or polypeptides that comprise a variant of SEQ ID NO: 2. In some aspects, the variants can comprise a sequence having at least 20%, 30%, 40%, 50%, 60%, 70%, 80%, 90%, 95%, 98%, 99% identity to SEQ ID NO: 2. In some aspects, the variant of SEQ ID NO: 2 has an amino acid sequence of at least 90% or 95% sequence identity when compared to SEQ ID NO: 2. In some aspects, the variant retains at least 50%, 75%, 80%, 85%, 90%, 95% or 99% of the biological activity of the reference protein described herein.

Generally, the amino acid identity between an individual variant peptides is at least 90%, 91%, 92%, 93%, 94%, 95%, 96%, 97%, 98%, 99%, or 100%. Thus, a “variant peptide” is one with the specified identity to the parent or reference peptide of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent peptide. For example, a “variant petpide” can be a sequence that contains two or more amino acid changes or more as compared to the parent or reference peptide of the invention, and shares or improves biological function, specificity and/or activity of the parent peptide.

In some aspects, any of peptide sequences disclosed herein can include a single amino acid change as compared to the parent or reference peptide sequence. In some aspects, any of the peptide sequences disclosed herein can include at least two amino acid changes as compared to the parent or reference peptide sequences. In some aspects, the amino acid change can be a change from a cysteine residue to another amino acid. In some aspects, the amino acid change can be a change from a glycine residue to another amino acid. The amino acid identity between individual variant peptide sequences can be at least 60%, 65%, 70%, 75%, 80%, 85%, 90%, 91%, 92%, 93%, 94%, 95%, 96%, 97%, 98%, 99% or 100%. Thus, a “variant peptide sequence” can be one with the specified identity to the parent peptide sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent peptide sequence. The variant peptide sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent peptide sequence.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, the variations in the amino acid sequence maintains at least 20%, more preferably at least 30%, 40%, 50%, 60%, 70%, 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

As used herein, the term “peptide” refers to a linear molecule formed by binding amino acid residues to each other via peptide bonds. As used herein, the term “polypeptide” refers to a polymer of (the same or different) amino acids bound to each other via peptide bonds.

In some aspects, the peptides, polypeptides, fragments or variants thereof disclosed herein are capable of recognizing, binding and removing one or more peroxins embedded in an outer mitochondrial membrane of a cell. In some aspects, the one or more peroxins can be a tail-anchored protein. In some aspects, the one or more peroxins is not a tail-anchored protein.

As used herein, the term “fragment of SEQ ID NO: 1” or “fragment of SEQ ID NO: 2” refers to a peptide or polypeptide designed to contain a partial peptide sequence comprising the amino acids residues at positions W166, Y167, R201, D205, and/or H206 of SEQ ID NO: 1 or SEQ ID NO: 2, wherein the fragment of SEQ ID NO: 1 or the fragment of SEQ ID NO: 2 can recognize, bind to one or more peroxins and remove one or more peroxins embedded in an outer mitochondrial membrane of a cell. In some aspects, the one or more peroxins can be a tail-anchored protein. In some aspects, the one or more peroxins is not a tail-anchored protein.

As used herein, the term “variant of SEQ ID NO: 1” or “variant of SEQ ID NO: 2” refers to a peptide or polypeptide designed to contain a partial peptide sequence comprising the amino acids residues at positions W166, Y167, R201, D205, and/or H206 of SEQ ID NO: 1 or SEQ ID NO: 2, wherein the variant of SEQ ID NO: 1 or the variant of SEQ ID NO: 2 can recognize, bind to one or more peroxins and remove one or more peroxins embedded in an outer mitochondrial membrane of a cell. In some aspects, the one or more peroxins can be a tail-anchored protein. In some aspects, the one or more peroxins is not a tail-anchored protein.

In some aspects, the peptide or polypeptide, fragments or variants can be of any length so long as the peptide or polypeptide, fragments or variants can recognize, bind to one or more peroxins and remove one or more peroxins embedded in an outer mitochondrial membrane of a cell.

In some aspects, the peptide or polypeptide, fragments or variants can be of any length so long as the peptide or polypeptide, fragments or variants can recognize, bind to one or more peroxins and remove one or more peroxins embedded in an outer mitochondrial membrane of a cell wherein the one or more peroxins is PEX13, PEX11, PEX2, PEX17, PEX3, PEX22, PEX25, PEX14, PEX4 or a combination thereof. In some aspects, the one or more peroxins can be can be a peroxisomal matrix protein. In some aspects, the peroxisomal matrix protein can be Pot1, Mdh3, Atg36 or a combination thereof. In some aspects, the one or more peroxins can be PEX13, PEX11, PEX2, PEX17, PEX3, PEX22, PEX25, PEX14, PEX4, Pot1, Mdh3, Atg36 or a combination thereof.

In some aspects, the peptides or polypeptides, fragments or variants described herein can further comprise 1, 2, 3, 4, 5, 10, 15, 20, 25, 30, 35, 40, 45, 50 additional amino acid residues at the N-terminal end of the disclosed peptides or polypeptides. In some aspects, the peptides or polypeptides, fragments or variants described herein can further comprise 1, 2, 3, 4, 5, 10, 15, 20, 25, 30, 35, 40, 45, 50 additional amino acid residues at the C-terminal end of the disclosed peptides or polypeptides disclosed herein. In some aspects, the additional amino acid residues that can be present at either the N-terminal end or the C-terminal end of any of the peptides, polypeptides, fragments or variants disclosed herein can be unimportant for recognizing, binding to one or more peroxins and removing one or more peroxins embedded in an outer mitochondrial membrane of a cell. In some aspects, the amino acid residues added to the N-terminal end or the C-terminal end of the peptides, polypeptides, fragments or variants disclosed herein may prevent ubiquitination, improve stability, help maintain the three dimensional structure of the peptide, or a combination thereof.

In some aspects, the peptides, polypeptides, fragments, or variants disclosed herein can further comprise a peptide or polypeptide having one or more amino acid residues with a modified side chain. In some aspects, one or more amino acids of any of the peptides, polypeptides, fragments or variants disclosed here can have a modified side chain. Examples of side chain modifications include but are not limited to modifications of amino acid groups, such as reductive alkylation; amidination with methylacetimidate; acylation with acetic anhydride; carbamolyation of amino groups with cynate; trinitrobenzylation of amino acid with 2,4,6-trinitrobenzene sulfonic acid (TNBS); alkylation of amino groups with succinic anhydride; and pyridoxylation with pridoxal-5-phosphate followed by reduction with NaBH₄.

In some aspects, the guanidine group of the arginine residue may be modified by the formation of a heterocyclic condensate using a reagent, such as 2, 3-butanedione, phenylglyoxal, and glyoxal. In some aspects, the carboxyl group may be modified by carbodiimide activation via O-acylisourea formation, followed by subsequent derivatization, for example, to a corresponding amide.

In some aspects, a sulfhydryl group can be modified by methods, such as carboxymethylation with iodoacetic acid or iodoacetamide; performic acid oxidation with cysteic acid; formation of mixed disulfides by other thiol compounds; a reaction by maleimide, maleic anhydride, or other substituted maleimide; formation of mercury derivatives using 4-chloromercuribenzoate, 4-chloromercuriphenylsulfonic acid, phenylmercury chloride, 2-chloromercuri-4-nitrophenol, and other mercurial agents; and carbamolyation with cyanate at alkaline pH. In addition, the sulfhydryl group of cysteine may be substituted with a selenium equivalent, whereby a diselenium bond may be formed instead of at least one disulfide bonding site in the peptide.

In some aspects, a tryptophan residue can be modified by, for example, oxidation with N-bromosuccinimide or alkylation of the indole ring by 2-hydroxy-5-nitrobenzyl bromide or sulfonyl halide. Meanwhile, the tyrosine residue may be modified by nitration using tetranitromethane to form a 3-nitrotyrosine derivative.

In some aspects, a modification of an imidazole ring of the histidine residue can be accomplished by alkylation with an iodoacetic acid derivative or N-carbethoxylation with diethylpyrocarbonate.

In some aspects, a proline residue can be modified by, for example, hydroxylation at the 4-position.

In some aspects, the peptides, polypeptides, fragments or variants described herein can be further modified to improve stability. In some aspects, any of the amino acid residues of the peptides, polypeptides, fragments or variants described herein can be modified to improve stability. In some aspects, peptides, polypeptides, fragments or variants can have at least one amino acid residue that has an acetyl group, a fluorenylmethoxy carbonyl group, a formyl group, a palmitoyl group, a myristyl group, a stearyl group, or polyethylene glycol. In some aspects, an acetyl protective group can be bound to the peptides, polypeptides, fragments or variants described herein.

As used herein, the term “stability” refers to storage stability (e.g., room-temperature stability) as well as in vivo stability. The foregoing protective group can protect the peptides, polypeptides, fragments or variants described herein from the attack of protein cleavage enzymes in vivo.

As used herein, the term “peptide or polypeptide” can also be used to include functional equivalents of the peptides or polypeptides described herein. As used herein, the term “functional equivalents” can refer to amino acid sequence variants having an amino acid substitution, addition, or deletion in some of the amino acid sequence of the peptide or polypeptide while simultaneously having similar or improved biological activity, compared with the peptide or polypeptide as described herein. In some aspects, the amino acid substitution can be a conservative substitution. Examples of the naturally occurring amino acid conservative substitution include, for example, aliphatic amino acids (Gly, Ala, and Pro), hydrophobic amino acids (Ile, Leu, and Val), aromatic amino acids (Phe, Tyr, and Trp), acidic amino acids (Asp and Glu), basic amino acids (His, Lys, Arg, Gln, and Asn), and sulfur-containing amino acids (Cys and Met). In some aspects, the amino acid deletion can be located in a region that is not directly involved in the activity of the peptide and polypeptide disclosed herein.

In some aspects, the amino acid sequence of the peptides, polypeptides, fragments or variants described herein can include a peptide sequence that has substantial identity to any of the sequences of the peptides or polypeptides disclosed herein. As used herein, the term “substantial identity” means that two amino acid sequences, when optimally aligned and then analyzed by an algorithm normally used in the art, such as BLAST, GAP, or BESTFIT, or by visual inspection, share at least about 60%, 70%, 80%, 85%, 90%, or 95% sequence identity. Methods of alignment for sequence comparison are known in the art.

In some aspects, the amino acid sequence of the peptides, polypeptides, fragments or variants described herein can include a peptide sequence that has some degree of identity or homology to any of sequences of the peptides or polypeptides disclosed herein. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The peptides or polypeptides described herein can have at least or about 25%, 50%, 65%, 75%, 80%, 85%, 90%, 95%, 96%, 97%, 98%, or 99% identity or homology to the peptide or polypeptide, wherein the peptide or polypeptide is one or more of SEQ ID NOs: 1-2.

In some aspects, the peptides or polypeptides described herein can be further conjugated to a label or detection tag (e.g., FLAG™ tag, epitope or protein tags, such as myc tag, 6 His, and fluorescent fusion protein). In some aspects, the label or detection tag can be a protein purification affinity tag. In some aspects, a label (e.g., FLAG™ tag) can be fused to the peptides or polypeptides disclosed herein. In some aspects, the disclosed methods and compositions further comprise a fusion protein, or a polynucleotide encoding the same. In various aspects, the peptides or polypeptides can comprise at least one epitope-providing amino acid sequence (e.g., “epitope-tag”), wherein the epitope-tag is selected from i) an epitope-tag added to the N- and/or C-terminus of the peptide or polypeptide; or ii) an epitope-tag inserted into a region of the peptide or polypeptide, and an epitope-tag replacing a number of amino acids in the peptide or polypeptide.

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In some aspects, the disclosed methods and compositions comprise an epitope-tag wherein the epitope-tag has a length of between 6 to 15 amino acids. In some aspects, the epitope-tag has a length of 9 to 11 amino acids. The disclosed methods and compositions can also comprise a peptide or polypeptide comprising two or more epitope-tags, either spaced apart or directly in tandem. Further, the disclosed methods and composition can comprise 2, 3, 4, 5 or even more epitope-tags, as long as the peptide or polypeptide maintains its biological or desired activity/activities (e.g., “functional”).

In some aspects, label, detection tag, epitope-tag, affinity tag or protein purification affinity tag can be His-tag, a FLAG-tag, a HA (hemagglutinin)-tag, a Strep-tag, a C9-tag, a glutathione S-transferase tag, a maltose-binding protein tag, a T7 tag, a V5 tag, an S tag, a SUMO tag, a TAP tag, a TRX tag, a calmodulin binding peptide, a chitin binding domain, a E2 epitope, a HaloTag, a HSV tag, a HBH tag, a KT3 tag, VSV-G tag, CD tag, Avitag, or GFP-tag or a myc-tag. The sequences of these tags are described in the literature and well known to the person of skill in art.

In some aspects, the peptides, polypeptides, fragments or variants disclosed herein can be used or administered in combination with other therapies used in the methods disclosed herein. In some aspects, any of the compositions or pharmaceutical compositions disclosed herein can further comprise a therapeutic agent. For example, in some aspects, any of the peptides disclosed herein can be administered before, or after or concurrently with one or more therapeutic agents. For instance, subjects with a perioxisomal biogenesis disorder can have a variety of symptoms including but not limited to seizures and spasticity that can be treated symptomatically. In some aspects, the therapeutic agent can be baclofen. Baclofen can be administered to reduce spasticity. In some aspects, the therapeutic agent can be keppra. Keppra can be administered to reduce seizure. In some aspects, baclofen or keppra can be administered enterally (e.g., oral or via g-tube) or intravenously.

Also disclosed herein are compositions comprising nucleic acids capable of encoding a peptide comprising or consisting of the amino acid sequence of SEQ ID NO: 1, or a fragment or variant thereof. Further disclosed herein are compositions comprising nucleic acids capable of encoding a peptide comprising or consisting of the amino acid sequence of SEQ ID NO: 2, or a fragment or variant thereof.

Also disclosed herein are vectors comprising nucleic acids capable of encoding a peptide comprising or consisting of the amino acid sequence of SEQ ID NO: 1 or SEQ ID NO: 2, or the fragments or the variants thereof. As used herein, a “vector” refers to a carrier molecule into which another DNA segment can be inserted to initiate replication of the inserted segment. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids and viruses. The term “vector” also includes expression vectors and refers to a vector containing a nucleic acid sequence coding for at least part of a gene product capable of being transcribed. A variety of ways can be used to introduce an expression vector into cells. In some aspects, the expression vector can comprise a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from bacteriophages and retroviruses. Vectors and expression systems are commercially available and known to one skilled in the art.

The vectors disclosed herein can also include detectable labels. Such detectable labels can include a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus.

The term “expression cassette” as used herein refers to a nucleic acid construct. The expression cassette can be produced either through recombinant techniques or synthetically that will result in the transcription of a certain polynucleotide sequence in a host cell. The expression cassette can be part of a plasmid, viral genome or nucleic acid fragment. Generally, the expression cassette includes a polynucleotide operably linked to a promoter. In some aspects, an expression cassette can comprise a promoter and the polynucleotide or polynucleotide sequence encoding any of peptides described herein. The expression cassette can further comprise a coding sequence for a nuclear localization signal fused to the N- or C-terminus of the polynucleotide and a polyadenylation signal. The expression cassette can further encode a detection or purification label or tag, fused to the N- or C-terminus of the polynucleotide. In some aspects, the expression cassette can be a plasmid. Plasmids that are useful include yeast plasmids, lentiviral plasmids and the like. The expression cassette can be adapted for expression in a specific type of host cell (e.g., using a specific type of promoter). The expression cassette can also comprise other components such as polyadenylation signals, enhancer elements or any other component that results in the expression of the peptides disclosed herein in a specific type of host cell.

As used herein, the term “operably linked” refers to the position of a regulatory region and a sequence to be transcribed in a nucleic acid to facilitate transcription or translation of the sequence. The choice of promoters depends on several factors including but not limited to efficiency, selectability, inducibility, desired expression level, and cell- or tissue-preferential expression. One skilled in the art is capable of appropriately selecting and positioning promoters and other regulatory regions relative to the coding sequence.

Vectors include, for example, viral vectors (such as adenoviruses (“Ad”), adeno-associated viruses (AAV), and retroviruses), liposomes and other lipid-containing complexes, and other macromolecular complexes capable of mediating delivery of a polynucleotide to a host cell. Vectors can also comprise other components to further modulate polynucleotide delivery and/or peptide expression, or that otherwise provides beneficial properties to the targeted cells. A wide variety of vectors is known to those skilled in the art and is generally available.

A host cell can be selected depending on the nature of the transfection vector. In some aspects, the host cell can comprise an expression cassette comprising a promoter and polynucleotide or polynucleotide sequence encoding any of peptides described herein. The promoter can be operably linked to the polynucleotide sequence encoding the peptide or protein. The cell can be examined using a variety of different physiologic assays. Such assays and methods are known to one skilled in the art.

The present disclosure also includes a nucleic acid comprising a sequence encoding any of the peptides, polypeptides, fragments or variants disclosed herein. As described above, the nucleic acids disclosed herein can be optimized for expression in an organism (e.g., human). The nucleic acid sequences disclosed herein can be codon optimized for efficient expression in mammalian cells (i.e., “humanized”).

Pharmaceutical Compositions

As disclosed herein, are pharmaceutical compositions, comprising a peptide, wherein the peptide comprises the sequence of SEQ ID NO: 1, or a variant or a fragment thereof, and a pharmaceutically acceptable carrier. Also disclosed herein, are pharmaceutical compositions, comprising a peptide, wherein the peptide comprises the sequence of SEQ ID NO: 1, or a variant or a fragment thereof, and a pharmaceutically acceptable carrier. Also disclosed herein, are pharmaceutical compositions, comprising a vector comprising a nucleic acid capable of encoding a peptide comprising the sequence of SEQ ID NO: 1, or a fragment or a variant thereof. Further disclosed herein are pharmaceutical compositions for removing one or more peroxins embedded in the outer mitochondrial membrane of a cell. Also disclosed herein are pharmaceutical compositions for reducing peroxin accumulation in the outer mitochondrial membrane of a cell. Further, disclosed herein are pharmaceutical compositions capable of rescuing mitochondrial function in a cell. In some aspects, the pharmaceutical compositions can further comprise a pharmaceutically acceptable carrier. In some aspects, the pharmaceutical compositions can comprise: a) a therapeutically effective amount of any of peptides or polypeptides described herein; and b) a pharmaceutically acceptable carrier. In some aspects, the peptides described herein can bind to or interact with one or more peroxins. In some aspects, the peptides described herein can recognize, bind and remove one or more peroxins embedded in an outer mitochondrial membrane of a cell.

The pharmaceutical compositions described above can be formulated to include a therapeutically effective amount of any of the peptides, polypeptides, fragments or variants described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a perioxisomal biogenesis disorder or Alzheimer's disease, Parkinson's disease, multiple sclerosis, or an age-related disease. In some aspects, the patient has a clinically determined predisposition or increased susceptibility (in some cases, a greatly increased susceptibility) for any disease with a false mitochondrial protein localization.

The pharmaceutical compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with a perioxisomal biogenesis disorder, Alzheimer's disease, Parkinson's disease, multiple sclerosis, or an age-related disease or any disease with false mitochondrial protein localization in an amount sufficient to at least partially improve a sign or symptom or to inhibit the progression of (and preferably arrest) the symptoms of the condition, its complications, and consequences (e.g., developing perioxisomal biogenesis disorder, Alzheimer's disease, Parkinson's disease, multiple sclerosis, or an age-related disease or any disease with false mitochondrial protein localization). In some aspects, the compositions can be administered to a subject (e.g., a human patient) already with or diagnosed with a perioxisomal biogenesis disorder, Alzheimer's disease, Parkinson's disease, multiple sclerosis, or an age-related disease or any disease with false mitochondrial protein localization in an amount sufficient to at least partially improve a sign or symptom or to inhibit the progression of (and preferably arrest) the symptoms of the condition, its complications, and consequences (e.g., perioxisomal biogenesis disorder, Alzheimer's disease, Parkinson's disease, multiple sclerosis, or an age-related disease or any disease with false mitochondrial protein localization). An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effect amount includes amounts that provide a treatment in which the onset or progression of a perioxisomal biogenesis disorder, Alzheimer's disease, Parkinson's disease, multiple sclerosis, or an age-related disease or any disease with false mitochondrial protein localization, peroxin accumulation in the outer mitochondrial membrane of a cell, or a deficiency in human ATAD1 levels or expression or a symptom of the perioxisomal biogenesis disorder, Alzheimer's disease, Parkinson's disease, multiple sclerosis, or an age-related disease or any disease with false mitochondrial protein localization, peroxin accumulation in the outer mitochondrial membrane of a cell, or a deficiency in human ATAD1 levels or expression is ameliorated. One or more of the symptoms can be less severe. In some aspects, recovery can be accelerated in an individual who has been treated with one or more of the compositions disclosed herein.

In some aspects, the pharmaceutical composition can be formulated for intravenous administration. In some aspects, the pharmaceutical composition can be formulated for direct injection. In some aspects, the pharmaceutical composition can be formulated for delivery to the cerebral spinal fluid. In some aspects, the pharmaceutical composition can be formulated for subcutaneous, intramuscular, intraperitonealy, intrathecally, intranasal, or oral administration. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the peptides or compositions disclosed herein. Thus, compositions can be prepared for parenteral administration that includes the peptides or polypeptides dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

The compositions described herein can be formulated with a carrier that can be pharmaceutically acceptable and that can be appropriate for delivering the peptide by the desired route of administration. Suitable pharmaceutically acceptable carriers can be those that are typically used with peptide-based drugs, such as diluents, excipients and the like. Reference can be made to “Remington's Pharmaceutical Sciences” by E. W. Martin, which is herein incorporated by reference, for guidance on drug formulations generally. In some aspects, the carrier can be selected based on its ability to solubilize and stabilize the peptide in solution. Further, the carrier can be selected based its ability to permit the release of the peptide into circulation after, for example, injection.

In some aspects, the compositions can be formulated for administration by infusion. In some aspects, the compositions can be formulated for administration by injection (e.g., subcutaneously, intramuscularly or intravenously) and can be used as aqueous solutions in sterile and pyrogen-free form and optionally buffered to physiologically tolerable pH, e.g., a slightly acidic or physiological pH. The compositions can be administered in a vehicle such as distilled water or in saline, phosphate buffered saline or 5% dextrose solution. Incorporating a solubility enhancer, such as acetic acid, can enhance water solubility of the compositions described herein.

The aqueous carrier or vehicle can be supplemented for use as injectables with an amount of gelatin that can serve to depot the peptide or polypeptides at or near the site of injection, for its slow release to the desired site of action. Concentrations of gelatin effective to achieve the depot effect can be in the range of 10-20%. Alternative gelling agents, such as hyaluronic acid, can also be useful as depositing agents.

In some aspects, any of the peptides, polypeptides, fragments or variants or compositions disclosed herein can be formulated as a slow release implantation device for extended and sustained administration. Examples of such sustained release formulations include but not limited to composites of biocompatible polymers, such as poly(lactic acid), poly(lactic-co-glycolic acid), methylcellulose, hyaluronic acid, collagen, and the like. Liposomes can also be used to provide for the sustained release of any of the peptides or compositions disclosed herein. Implantable osmotic minipumps can also be used for sustained release. Sustained release formulations can provide a high local concentration of any of the peptides or compositions disclosed herein. In some aspects, the compositions described herein can be formulated for sustained release.

The compositions disclosed herein can be used in the form of a sterile-filled vial or ampoule that can contain a desired amount of the peptide in either unit dose or multi-dose amounts. The vial or ampoule can contain the any of the peptides or compositions disclosed herein and the desired carrier as an administration-ready formulation. Alternatively, the vial or ampoule can contain the any of the peptides or compositions disclosed herein in a form, such as a lyophilized form, suitable for reconstitution in a suitable carrier, such as phosphate-buffered saline.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. In some aspects, the pharmaceutically acceptable carrier can be a solvent, dispersion media, coatings, antibacterial, isotonic and absorption delaying agents, buffers, excipients, binders, lubricants, gels, surfactants that can be used as media for a pharmaceutically acceptable substance. In some aspects, can be a lipid-based or polymer-based colloid. Examples of colloids include liposomes, hydrogels, microparticles, nanoparticles and micelles. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

Methods of Treatment

Disclosed herein are methods of removing one or more peroxins embedded in the outer mitochondrial membrane of a cell in a subject. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein removes one or more peroxins embedded in the outer mitochondrial membrane of a cell in the subject.

Disclosed herein are methods of reducing peroxin accumulation in the outer mitochondrial membrane of a cell in a subject. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein reduces peroxin accumulation in the outer mitochondrial membrane. In some aspects, the therapeutically effective amount of the composition, pharmaceutical composition, or peptides, polypeptides, fragments or variants disclosed herein removes one or more peroxins embedded in the outer mitochondrial membrane of a cell in the subject.

Disclosed herein are methods of rescuing mitochondrial function in a cell of a subject. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein rescuing mitochondrial function in a cell of the subject. In some aspects, the therapeutically effective amount of the composition, pharmaceutical composition, or peptides, polypeptides, fragments or variants disclosed herein removes one or more peroxins embedded in the outer mitochondrial membrane of a cell in the subject.

Disclosed herein are methods of restoring mitochondrial respiration in a subject. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides disclosed herein can restore mitochondrial respiration in the subject. In some aspects, the therapeutically effective amount of the composition, pharmaceutical composition, or peptides, polypeptides, fragments or variants disclosed herein removes one or more peroxins embedded in the outer mitochondrial membrane of a cell in the subject.

Disclosed herein are methods of restoring mitochondrial respiration in a cell. In some aspects, the methods comprise: contacting a cell with a therapeutically effective amount of any of the compositions, pharmaceutical compositions, or peptides, polypeptides, fragments or variants disclosed herein. In some aspects, the therapeutically effective amount of the composition, pharmaceutical composition, or peptides, polypeptides, fragments or variants disclosed herein can remove one or more peroxins embedded in the outer mitochondrial membrane of a cell in a subject. In some aspects, the therapeutically effective amount of the composition, pharmaceutical composition, or peptides, polypeptides, fragments or variants disclosed herein can reduce peroxin accumulation in the outer mitochondrial membrane of a cell in a subject. In some aspects, the therapeutically effective amount of the composition, pharmaceutical composition, or peptides, polypeptides, fragments or variants disclosed herein can rescue mitochondrial function in a cell of a subject. In some aspects, the cell can be in the subject.

Disclosed herein are methods of treating a subject that has or has been diagnosed with a perioxisomal biogenesis disorder. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein removes one or more peroxins embedded in the outer mitochondrial membrane of a cell in the subject, thereby treating the subject. In some aspects, the therapeutically effective amount of the composition, pharmaceutical composition, or peptides, polypeptides, fragments or variants disclosed herein reduces peroxin accumulation in the outer mitochondrial membrane of a cell in the subject, thereby treating the subject. In some aspects, the therapeutically effective amount of the composition, pharmaceutical composition, or peptides disclosed herein rescues mitochondrial function in cell of the subject, thereby treating the subject.

Disclosed herein are methods of reducing one or more symptoms of a perioxisomal biogenesis disorder in a subject. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein removes one or more peroxins embedded in the outer mitochondrial membrane of a cell in the subject. In some aspects, the therapeutically effective amount of the composition, pharmaceutical composition, or peptides, polypeptides, fragments or variants disclosed herein reduces peroxin accumulation in the outer mitochondrial membrane of a cell in the subject. In some aspects, the therapeutically effective amount of the composition, pharmaceutical composition, or peptides, polypeptides, fragments or variants disclosed herein rescues mitochondrial function in a cell of the subject, thereby treating the subject. In some aspects, the therapeutically effective amount of the composition, pharmaceutical composition, or peptides, polypeptides, fragments or variants disclosed herein reducing one or more symptoms of a perioxisomal biogenesis disorder in the subject. In some aspects, the one or more symptoms of a perioxisomal biogenesis disorder can be neonatal seizures, hepatomegaly, renal cysts, skeletal abnormalities, impaired hearing, weakness, poor eyesight, shortness of breath or a combination thereof.

Disclosed herein are methods of treating a subject with a deficiency in human ATAD1 levels or expression. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein removes one or more peroxins embedded in the outer mitochondrial membrane of a cell in the subject, thereby treating the subject. In some aspects, the therapeutically effective amount of the composition, pharmaceutical composition, or peptides, polypeptides, fragments or variants disclosed herein reduces peroxin accumulation in the outer mitochondrial membrane of a cell in the subject, thereby treating the subject. In some aspects, the therapeutically effective amount of the composition, pharmaceutical composition, or peptides, polypeptides, fragments or variants disclosed herein rescues mitochondrial function in a cell of the subject, thereby treating the subject.

Disclosed herein are methods of increasing human ATAD1 protein or gene levels or expression in a subject. In some aspects, the methods comprise: administering to a subject a therapeutically effective amount of any of the compositions, pharmaceutical compositions, or peptides, polypeptides, fragments or variants disclosed herein. In some aspects, the methods can further comprise: administering a therapeutically effective amount of any of the compositions, pharmaceutical compositions, or peptides, polypeptides, fragments or variants disclosed herein and a pharmaceutically acceptable carrier. In some aspects, the therapeutically effective amount of the composition, pharmaceutical composition, or peptides, polypeptides, fragments or variants disclosed herein removes one or more peroxins embedded in the outer mitochondrial membrane of a cell in the subject, thereby treating the subject. In some aspects, the therapeutically effective amount of the composition, pharmaceutical composition, or peptides, polypeptides, fragments or variants disclosed herein reduces peroxin accumulation in the outer mitochondrial membrane of a cell in the subject, thereby treating the subject. In some aspects, the therapeutically effective amount of the composition, pharmaceutical composition, or peptides, polypeptides, fragments or variants disclosed herein rescues mitochondrial function in a cell of the subject, thereby treating the subject.

Disclosed herein are methods of treating a deficiency in human ATAD1 levels or expression in a cell. In some aspects, the methods comprise: contacting a cell with a therapeutically effective amount of any of the compositions, pharmaceutical compositions, or peptides, polypeptides, fragments or variants disclosed herein. In some aspects, the therapeutically effective amount of the composition, pharmaceutical composition, or peptides, polypeptides, fragments or variants disclosed herein can remove one or more peroxins embedded in the outer mitochondrial membrane of a cell in a subject. In some aspects, the therapeutically effective amount of the composition, pharmaceutical composition, or peptides, polypeptides, fragments or variants disclosed herein can reduce peroxin accumulation in the outer mitochondrial membrane of a cell in a subject. In some aspects, the therapeutically effective amount of the composition, pharmaceutical composition, or peptides, polypeptides, fragments or variants disclosed herein can rescue mitochondrial function in a cell of a subject. In some aspects, the cell can be in the subject.

Disclosed herein are methods of increasing human ATAD1 protein or gene levels or expression in a cell. In some aspects, the methods comprise: contacting a cell with a therapeutically effective amount of any of the compositions, pharmaceutical compositions, or peptides, polypeptides, fragments or variants disclosed herein. In some aspects, the therapeutically effective amount of the composition, pharmaceutical composition, or peptides, polypeptides, fragments or variants disclosed herein can remove one or more peroxins embedded in the outer mitochondrial membrane of a cell in a subject. In some aspects, the therapeutically effective amount of the composition, pharmaceutical composition, or peptides, polypeptides, fragments or variants disclosed herein can reduce peroxin accumulation in the outer mitochondrial membrane of a cell in a subject. In some aspects, the therapeutically effective amount of the composition, pharmaceutical composition, or peptides, polypeptides, fragments or variants disclosed herein can rescue mitochondrial function in a cell of a subject. In some aspects, the cell can be in the subject.

Disclosed herein are methods of inhibiting the import of one or more peroxisomal matrix proteins into the mitochondria of a cell. In some aspects, the methods comprise: contacting a cell with a therapeutically effective amount of any of the compositions, pharmaceutical compositions, or peptides, polypeptides, fragments or variants disclosed herein. In some aspects, peptide of the composition or pharmaceutical composition can be capable of binding to the peroxisomal matrix protein. In some aspects, the one or more peroxisomal matrix proteins can be POT1, MDH3, or ATG36. In some aspects, the peptides, polypeptides, fragments or variants of the composition or pharmaceutical composition can indirectly inhibit (or abolish) the import of one or more peroxisomal matrix proteins into the mitochondria of the cell. In some aspects, the methods of inhibiting the import of one or more peroxisomal matrix proteins into the mitochondria of a cell can be by direct or indirect mechanism(s). For example, when one or more peroxisomes are absent, peroxins can accumulate on the outer mitochondrial membrane. The assembly which can occur containing those peroxins might be involved in peroxisomal matrix protein import into the mitochondria. If the peroxin accumulation is removed, the formation of a peroxin assembly can be avoided or inhibited or blocked which in turn reduces the amount of peroxisomal matrix proteins found in the mitochondria, and, as such have a beneficial or therapeutic effect. In some aspects, the removal of one or more peroxins by ATAD1 (or by overexpressing ATAD1) might be mediated by an indirect process in which the peroxin is not in contact with ATAD1 specifically, but rather by recruiting e.g., ESCRT proteins to the mitochondrial membrane which would polarize, bud a vesicle which then becomes siccioned for release. The siccioning process can involve ATAD1 acting on a substrate resulting in removal of one or more peroxins without direct interaction of ATAD1 with the peroxin.

In some aspects, the therapeutically effective amount of the composition, pharmaceutical composition, or peptides, polypeptides, fragments or variants disclosed herein can remove one or more peroxins embedded in the outer mitochondrial membrane of a cell in a subject. In some aspects, the therapeutically effective amount of the composition, pharmaceutical composition, or peptides, polypeptides, fragments or variants disclosed herein can reduce peroxin accumulation in the outer mitochondrial membrane of a cell in a subject. In some aspects, the therapeutically effective amount of the composition, pharmaceutical composition, or peptides, polypeptides, fragments or variants disclosed herein can rescue mitochondrial function in a cell of a subject.

In some aspects, the cell can be in the subject. In some aspects, the cell can be any mammalian cell that contains one or more mitochondria. In some aspects, the cell can be a fibroblast, a hepatocyte, a neuron, or a muscle cell.

In some aspects, in any of the methods disclosed herein, the therapeutically effective amount of the composition, pharmaceutical composition, peptides, polypeptides, fragments or variants can be administered orally, intramuscularly, intraperitonealy, intravenously, subcutaneously or intrathecally.

In some aspects, the subject can be a human. In some aspects, the subject can be a human patient. In some aspects, the subject has a perioxisomal biogenesis disorder. In some aspects, the perioxisomal biogenesis disorder can be Zellweger Syndrome, neonatal adrenoleukodystrophy, or infantile Refsum disease. In some aspects, the subject has Alzheimer's disease, Parkinson's disease, multiple sclerosis, or an age-related disease. In some aspects, any of the methods disclosed herein can improve mitochondrial quality control. In some aspects, the subject can have any disease with a false mitochondrial protein localization. In some aspects, the subject has a deficiency in ATAD1 protein or gene levels or expression. In some aspects, the subject does not have a deficiency in ATAD1 protein or gene levels or expression. In some aspects, the subject has a perioxisomal biogenesis disorder and does not have a deficiency in ATAD1 protein or gene levels or expression when compared to a subject that does not have a perioxisomal biogenesis disorder.

In some aspects, the one or more peroxins can be embedded in the outer mitochondrial membrane. In some aspects, the one or more peroxins can be PEX13, PEX11, PEX2, PEX17, PEX3, PEX22, PEX25, PEX14, PEX4 or a combination thereof. In some aspects, the one or more peroxins can be can be a peroxisomal matrix protein. In some aspects, the peroxisomal matrix protein can be Pot1, Mdh3, Atg36 or a combination thereof. In some aspects, the one or more peroxins can be PEX13, PEX11, PEX2, PEX17, PEX3, PEX22, PEX25, PEX14, PEX4, Pot1, Mdh3, Atg36 or a combination thereof.

In some aspects, in any of the methods disclosed herein the peptides, polypeptides, fragments or variants of the composition or pharmaceutical composition can indirectly remove one or more peroxins from the mitochondria of the cell. In some aspects, the removal of the one or more peroxins embedded in the outer mitochondrial membrane of a cell can be by a vesicular mechanism. In some aspects, ATAD1 may act on ESCRT-proteins which allows part of the mitochondrial membrane to polarize, bud a vesicle which then becomes siccioned for release by ATAD1's activity, thereby allowing for simultaneous release of one or more peroxins.

In some aspects, a subject can have a deficiency in human ATAD1 protein levels or gene expression. In some aspects, the amount (protein levels or gene expression) of ATAD1 in a sample can be decreased or lower when compared to the amount in a control or a reference sample. In some aspects, a sample from a subject can be identified as being in need of treatment when the amount of ATAD1 (protein or gene) in the sample is decreased or lower when compared to the amount of ATAD1 (protein or gene) in a control or reference sample. In some aspects, the sample from the subject can be identified as having or being at risk for developing a perioxisomal biogenesis disorder or any disease with a false mitochondrial protein localization. In some aspects, the control or reference sample can be from an age-matched sample. In some aspects, the sample can be from one or more subjects that do not have or are known to not be at risk for developing a perioxisomal biogenesis disorder or any disease with a false mitochondrial protein localization.

As used herein, the terms, “reference,” “reference expression,” “reference sample,” “reference value,” “control,” “control sample” and the like, when used in the context of a sample or expression level of the amount of ATAD1 refers to a reference standard wherein the reference is expressed at a constant level, and is unaffected by the experimental conditions, and is indicative of the level in a sample of a predetermined disease status (e.g., not suffering from a perioxisomal biogenesis disorder or any disease with a false mitochondrial protein localization) or whether a subject (or disease) will respond to a therapeutic agent or treatment. The reference value can be a predetermined standard value or a range of predetermined standard values, representing no illness, or a predetermined type or severity of illness or representing the likelihood a disease, disorder or condition will be responsive to a particular type of therapeutic agent or treatment.

Reference expression can be the level or amount of ATAD1 in a reference sample from a subject, or a pool of subjects, not suffering from disease, disorder or condition (e.g., a perioxisomal biogenesis disorder or any disease with a false mitochondrial protein localization), with a known response (or lack thereof) to a particular treatment or known to be at risk for having or developing a disease, disorder or condition (e.g., a perioxisomal biogenesis disorder or any disease with a false mitochondrial protein localization). In some aspects, the reference value can be taken a different time point than to which it is being compared.

As used herein, a “reference value” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the individual before administration of or exposure to a particular therapeutic agent, but at an earlier point in time, or a value obtained from a sample from a subject other than the individual being tested, or a “normal” individual, that is an individual not diagnosed with any of the diseases, disorders or conditions described herein. The reference value can be based on a large number of samples, such as from subjects with any of the diseases, disorders or conditions described herein or normal individuals or based on a pool of samples including or excluding the sample to be tested. The reference value can also be based on a sample from subjects with any of the diseases, disorders or conditions described herein other than the individual being tested, or a “normal” individual that is an individual not diagnosed with any of the diseases, disorders or conditions described herein that has not or has been administered or exposed to a particular therapeutic agent.

The reference level used for comparison with the measured level of ADAT1 can vary, depending the method begin practiced, as will be understood by one of ordinary skill in the art. For methods for determining the likelihood a disease, disorder or condition (e.g., a perioxisomal biogenesis disorder or any disease with a false mitochondrial protein localization), a subject or a sample will be responsive to a particular type of therapeutic agent or treatment, the “reference level” is typically a predetermined reference level, such as an average of levels obtained from a population that has either been exposed or has not been exposed to particular type of therapeutic agent or treatment, but in some instances, the reference level can be a mean or median level from a group of individuals that are responders or non-responders. In some instances, the predetermined reference level can be derived from (e.g., is the mean or median of) levels obtained from an age-matched population.

Age-matched populations (from which reference values may be obtained) can be populations that are the same age as the individual being tested, but approximately age-matched populations are also acceptable. Approximately age-matched populations may be within 1, 2, 3, or 4 weeks or 1, 2, 3, 4, 5, 6, 7, 8, 9, 10, or 11 months or 1, 2, 3, 4, or 5 years of the age of the individual tested, or may be groups of different ages which encompass the age of the individual being tested. Approximately age-matched populations may be in 2, 3, or 4 week increments, 2, 3, 4, 5, 6, 7, 8, 9, 10, or 11 month increments or 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals or a “2 year increment” group which serves as the source for reference values for a 6 year old individual might include 4-6 year old individuals). In some aspects, the age-matched population can be a pediatric population.

Determining the level or amount of ADAT1 can include determining whether the level or amount of ADAT1 is decreased as compared to a control or reference sample or a sample that has been contacted, administered or exposed to a particular therapeutic agent or treatment, decreased compared to a control or reference sample or a sample that has been contacted, administered or exposed to a particular therapeutic agent or treatment, or unchanged compared to a control or reference sample or a sample that has been contacted, administered or exposed to a particular therapeutic agent or treatment. As used herein, the terms, “increased” or “increased expression level” or “increased level of expression” or “increased amount of protein” or “high” or “higher level” or “higher expression level” refers to an amount of ADAT1 that is expressed wherein the measure of the quantity of ADAT1 exhibits an increased level of expression when compared to a reference sample or “normal” control or a sample that has been contacted, administered or exposed to a particular therapeutic agent or treatment. An “increased expression level” or “higher expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “decreased,” “decreased level of expression,” or “decreased expression level” or “decreased amount of protein” or “low” or “lower level” or “lower expression level” refers to an amount of ADAT1 that is expressed wherein the measure of the quantity of the ADAT1 exhibits a decreased level of expression when compared to a reference sample or “normal” control or a sample that has been contacted, administered or exposed to a particular therapeutic agent or treatment. A “decreased level of expression” or “lower expression level” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

The level of ADAT1 can be a measure, for example, per unit weight or volume. In some aspects, the expression level can be a ratio (e.g., the amount of ADAT1 in a sample relative to the amount of the ADAT1 of a reference value or in a reference sample that may have been or may have not been also contacted with a therapeutic agent).

The method of comparing a measured value and a reference value or a measured value before and after contact with a therapeutic agent can be carried out in any convenient manner appropriate to the type of measured value. For example, ‘measuring’ can be performed using quantitative or qualitative measurement techniques, and the mode of comparing a measured value and a reference value can vary depending on the measurement technology employed. For example, the measured values used in the methods described herein can be quantitative values (e.g., quantitative measurements of concentration, such as nanograms of the ADAT1 per milliliter of sample, or absolute amount). As with qualitative measurements, the comparison can be made by inspecting the numerical data, by inspecting representations of the data (e.g., inspecting graphical representations such as bar or line graphs).

In some aspects, the level or amount of ADAT1 can be determined by various analysis methods. For example, the level or amount of ADAT1 can be determined in various immunoassay formats. These immunological analysis methods may be carried out according to various quantitative immunoassay protocols that have been developed in the prior art. Examples of the immunoassay format include radioactive immunoassay, radioactive immunoprecipitation, immunoprecipitation, enzyme-linked immunosorbent assay (ELISA), captured-ELISA, inhibition or competition analysis, sandwich assay, immunofluorescent staining, and immunoaffinity purification, but are not limited thereto.

In some aspects, the subject can be identified as being in need of treatment before the administration step. In some aspects, the subject can have a perioxisomal biogenesis disorder or any disease with an inappropriate mitochondrial protein localization or a combination thereof. In some aspects, the subject can have Alzheimer's disease, Parkinson's disease, multiple sclerosis, or an age-related disease. In some aspects, the subject can have Zellweger Syndrome, neonatal adrenoleukodystrophy, or infantile Refsum disease.

Amounts effective for these uses can depend on the severity of the condition, disease or disorder or the severity of the risk of the condition, disease or disorder, and the weight and general state and health of the subject, but generally range from about 0.05 μg to about 1000 μg (e.g., 0.5-100 μg) or 0.5 mg/kg to about 3 mg/kg per body weight per subject of an equivalent amount of the peptide per dose per subject. Suitable regimes for initial administration and booster administrations are typified by an initial administration followed by repeated doses at one or more hourly, daily, weekly, or monthly intervals by a subsequent administration. For example, a subject can receive any of polynucleotides, peptides or polypeptides or fragments or variants described herein in the range of about 0.05 to 1,000 μg or 0.5 to 3.0 mg/kg body weight equivalent dose per dose one or more times per week (e.g., 2, 3, 4, 5, 6, or 7 or more times per week). For example, a subject can receive 0.1 to 2,500 μg (e.g., 2,000, 1,500, 1,000, 500, 100, 10, 1, 0.5, or 0.1 μg) dose per week. In some aspects, a subject can receive 0.5 to 3.0 mg/kg daily (e.g., 3.5 to 21 mg/kg body weight). In some aspects, a subject can receive 0.5 to 3.0 mg/kg (e.g., 3.5 to 21 mg/kg body weight) daily dose per week. In some aspects, a subject can receive 0.5 to 3.0 mg/kg (e.g., 3.5 to 21 mg/kg body weight) per dose for one or more does per day per week. A subject can also receive any of the peptides or polypeptides described herein in the range of 0.1 to 3,000 μg per dose once every two or three weeks. In some aspects, a subject can also receive any of the polynucleotides, peptides or polypeptides described herein in the range of 0.5 to 3.0 mg/kg body weight per dose once every two or three weeks. A subject can also receive 2 mg/kg every week (with the weight calculated based on the weight of the polynucleotide, peptide or polypeptide described herein and the weight in kg calculated based on the weight of the subject).

In some aspects, the compositions comprising nucleic acids can be administered as a single dose or repeated. In some aspects, the nucleic acid capable of encoding a peptide comprising the sequence of SEQ ID NO: 1 or SEQ ID NO: 2, or fragments or variants thereof upon or after administration to a subject can remain in the subject's body and can continue to be expressed. In some aspects, the nucleic acid can be molecular construct, such as DNA or RNA, and is expressed under the control of a promoter or other mechanism so that the molecular construct can express a therapeutically effective amount of, for example, ATAD1 protein. In some aspects, the compositions disclosed herein can be administered intravenously or via cerebrospinal fluid, or via other approaches. The level/amount of gene or protein expression can be determined and adjusted to provide a therapeutic amount specific for the disease condition.

The total effective amount of the polynucleotides, peptides, polypeptides, fragments or variants in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

Duration of the treatment with any of the polynucleotides, peptides, polypeptides, fragments or variants or compositions as disclosed herein can be any length of time as short as 1 s, 10 s, 15 s, 30 s, 40 s, 50 s, or 60 s to as long as 1 month, 2 months, 3 months, 5 months or 6 months. In some aspects, the treatment with peptides or compositions as disclosed herein can be 1 minute, 5 minutes, 10 minutes, 15 minutes, 30 minutes, 1 hour, 2 hours, 3 hours, 4 hours, 5 hours, 10 hours, 15 hours, 20 hours, 21 hours, 22 hours, 23 hours, 24 hours, 2 days, 3 days, 4 days, 5 days, 10 days, 15, days, 20 days, 30 days, 2 months, 3 months, 4 months, 5 months, 6 months or any time in between or longer. For example, the peptides or compositions disclosed herein can be administered 1 second, 2 seconds, 3 seconds, 4 seconds, 5 seconds, 10 seconds, 15 seconds, 30 seconds, 1 minute, 5 minutes, 10 minutes, 15 minutes, 30 minutes, 1 hour, 2 hours, 3 hours, 4 hours, 5 hours, 10 hours, 15 hours, 20 hours, 21 hours, 22 hours, 23 hours, 24 hours or any time (seconds, minutes, hours) in between before the administration of rehydration therapy. The frequency of the treatment can vary. In some aspects, the initial administration of any of the polynucleotides, peptides, polypeptides, fragments or variants or compositions disclosed herein can precede the initial administration of any of the other therapeutic agents administered to the subject by 1 second, 2 seconds, 3 seconds, 4 seconds, 5 seconds, 10 seconds, 15 seconds, 30 seconds, 1 minute, 5 minutes, 10 minutes, 15 minutes, 30 minutes, 1 hour, 2 hours, 3 hours, 4 hours, 5 hours, 10 hours, 15 hours, 20 hours, 21 hours, 22 hours, 23 hours, 24 hours or any time (seconds, minutes, hours) in between or longer. In some aspects, the subsequent administration(s) of any of the peptides or compositions disclosed herein can be for part of or for the whole duration of the days that the subject receives any other therapeutic agent(s).

The therapeutically effective amount of any of the polynucleotides, peptides, polypeptides, fragments or variants present within the compositions described herein and used in the methods as disclosed herein applied to mammals (e.g., humans) can be determined by one of ordinary skill in the art with consideration of individual differences in age, weight, and other general conditions (as mentioned above).

Articles of Manufacture

The peptides, polypeptides, fragments or variants or the compostions or pharmaceutical compositions described herein can be packaged in a suitable container labeled, for example, for use to remove one or more peroxins embedded in the outer mitochondrial membrane of a cell, to reduce peroxin accumulation in the outer mitochondrial membrane of a cell, to rescue mitochondrial function in a cell, to restore mitochondrial respiration in a subject or a cell, to import one or more peroxisomal matrix proteins into the mitochondria of a cell in subjects with a perioxisomal biogenesis disorder, Alzheimer's disease, Parkinson's disease, multiple sclerosis, or an age-related disease, or any disease with a false mitochondrial protein localization; to treat a subject with a deficiency in human ATAD1 levels or expression; to treat a subject that does not have a deficiency in human ATAD1 levels or expression; and/or to ameliorate or reduce one or more symptoms of a perioxisomal biogenesis disorder, Alzheimer's disease, Parkinson's disease, multiple sclerosis, or an age-related disease, or any disease with a false mitochondrial protein localization. Accordingly, packaged products (e.g., sterile containers containing the composition described herein and packaged for storage, shipment, or sale at concentrated or ready-to-use concentrations) and kits, including at least any of the peptides as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The peptides or compositions disclosed herein can be ready for administration (e.g., present in dose-appropriate units), and may include a pharmaceutically acceptable adjuvant, carrier or other diluent. Alternatively, the peptides or compositions can be provided in a concentrated form with a diluent and instructions for dilution.

### EXAMPLES

### Example 1: ATAD1 Plays a Role in Mitochondrial Quality Control and Rescues the Phenotype in Human PBD/ZSD Fibroblasts

Abstract. Peroxisomal Biogenesis Disorders (PBDs) are inherited metabolic disorders with significant neurological sequelae. Peroxin mutations affect the import capacity into the peroxisomal matrix or lead to inactive peroxisomal remnants. To date the question of what happens to the peroxisomal proteins when the organelle no longer exists or can't import proteins has not explored. As described herein, the destiny of peroxins in PBDs was traced. First, the fate of peroxins was explored on a genome level leading to the mechanism(s) on the protein level. The results show that interfering with peroxisomal biogenesis surprisingly maintained peroxin gene expression. Subsequently, it led to the discovery that peroxins (peroxisomal biogenesis factors) accumulate in the mitochondrial membrane affecting respiration and ATP generation. Next, mitochondrial quality control was investigated, and the results show that mitochondrial function can be rescued in cells of human patients with Zellweger Syndrome, the most severe PBD, by overexpression of ATAD1, an AAA-ATPase functioning in protein quality control. Thus, the data described herein provide evidence for a biochemical coping strategy of the cell in the case of peroxisomal dysfunction. This finding can serve as an important target to improve the phenotype of PBDs and therefore improve and prolong the life of, for example, pediatric patients.

Introduction. Peroxisomes are present in most eukaryotes and are surrounded by a single membrane. These organelles function in fatty acid metabolism and scavenging of reactive oxygen species, pathways which are shared with the mitochondria. The mammalian peroxisome is involved in the biosynthesis of bile acids, purine, and specific lipids. One class of lipids which is solely synthesized in peroxisomes is plasmalogens, lipids which act as a precursor to forming the myelin sheath of neurons. Peroxisomal membrane proteins (PMPs) are primarily comprised of metabolite transporters and peroxins. Peroxins are peroxisome biogenesis factors that participate in different aspects of peroxisomal biogenesis, including protein import, inheritance, and division. More than 50 different enzymes are imported into the matrix of peroxisomes, where the majority of peroxisomal reactions occur. Given the multitude of metabolic pathways peroxisomes are involved in, it comes as no surprise that a defect in peroxisomal function or biogenesis has a severe phenotype. Mutations in most of the genes encoding peroxins are linked to peroxisomal biogenesis disorders (PBDs) with various degrees of severity. PBDs are inherited metabolic and neuropathic diseases that affect a multitude of metabolic pathways, often leading to death in early childhood.

PBDs include Zellweger Syndrome (ZSD), neonatal adrenoleukodystrophy and infantile Refsum disease. ZSD has the most severe presentation of the PDBs, with myriad clinical features including: neonatal seizures, hepatomegaly, renal cysts, skeletal abnormalities, impaired hearing, and symptoms related to mitochondrial abnormalities such as weakness, poor eyesight and shortness of breath.

In diseases in which sorting systems such as the GET system are impaired mislocalization of proteins challenges the cells quality control. Membrane proteins which cannot go to their intended destination need to find another membrane, if not, they are degraded. Common organelles which are affected by mislocalization are the endoplasmatic reticulum and the mitochondria. At least some peroxisomal proteins such as Pex15 can become targeted to the mitochondria when certain protein-sorting pathways are impaired. Yeast Msp1 and its mammalian homologue ATAD1, which belong to the AAA+ATPase protein family, facilitate the extraction and degradation of mislocalized tail-anchored proteins from mitochondria. Msp1/ATAD1 specialize in extracting its substrates from membranes. This suggests that Msp1 may help regulate peroxin localization when peroxisomes are absent, and, further, that the mitochondrial dysfunctions characteristic of PBDs could be the direct result of peroxin buildup in mitochondrial membranes. To this end, single and double deletions of pex3 and msp1 as well as pex19 and msp1 were generated. Targeting this quality control mechanism could be beneficial in the context of mitochondrial protein accumulation and disassembly of complexes.

Further evidence supporting the focus on mitochondria and its quality control is that mitochondrial dysfunction is indeed a widely recognized phenotype in PBD. Peroxisomes and mitochondria are functionally and physically engaged. Mitochondria share and complement peroxisomal metabolic pathways of lipid metabolism and reactive oxygen species defense as well as contributing to peroxisomal biogenesis. Recently, a pathway was proposed in which peroxins traffic to the mitochondria and into mitochondrial derived vesicles, which fuse with ER-derived preperoxisomes and give rise to functional peroxisomes.

Mitochondria overall serve to fulfill the metabolic need of the cells, of which some of them are particularly depended on for their function. Commonly not appreciated, retina cells have the greatest metabolic demand in the body. Also, the well-balanced coupling of mitochondrial function with neuronal activity ensures the execution of complex processes such as neurotransmission and plasticity in neurons. The extent in which the loss of mitochondrial function is exacerbating symptoms such as e.g., vision loss of PBDs, is not clear. Historically, the first Zellweger Syndrome patients were actually diagnosed because of their mitochondrial abnormalities. Despite these reports, the reasoning behind these findings as well as the underlaying cell biology is still elusive. Therefore, it was tested whether the escaped mechanistic evaluation of the destiny of peroxins will contribute to clarify PBD phenotypes as well as the cell biological coping strategy.

To explore the fate of peroxins in absence of peroxisomes and the resulting challenges for protein quality control, the yeast S. cerevisae was used because it is easy to genetically modulate and abolish peroxisomal biogenesis by deletion of pex3 and pex19. This genetic modulation causes the elimination of active peroxisomes in the living yeast cell. This allows the investigation of feedback signaling and its consequences. Acknowledging the main feature of PBD, as being the absence of peroxisomes, it was examined whether this radical change influences gene expression on a transcripitional/translational level before exploring the cause of the mitochondrial phenotype on protein level.

As described herein, it was discovered that the absence of peroxisomes does not interfere with peroxin RNA levels nor the translational efficiency of peroxisomal proteins. The finding that peroxins seem not to be regulated on a transcriptional or translational level led to the exploration of the fate of peroxisomal proteins in the absence of their native destination. This exploration unraveled a mechanism that causes mitochondrial dysfunction in Zellweger Syndrome. Peroxisomal membrane proteins accumulate on mitochondrial membranes and assemble into complexes with their peroxisome-native binding partners. While confirming this accumulation in human patient fibroblasts, it was found this accumulation interferes with mitochondrial respiration and therefore ATP production. Furthermore, it was observed that overexpression of human ATAD1 reduces peroxin accumulation on mitochondria and rescues mitochondrial function in fibroblasts from human patients with Zellweger Syndrome. Taken together, these results suggest that the severity of the neurological symptoms in PBD might improve upon restoring mitochondrial function. The results described herein provide information about the cell biology of mitochondria in the absence of peroxisomes and highlights the possibility to improve, PBDs as well as any disease which displays protein accumulation in the outer mitochondrial membrane. As described herein, improving mitochondrial quality control can provide a therapeutic strategy for any disease with false mitochondrial protein localization, like PBDs.

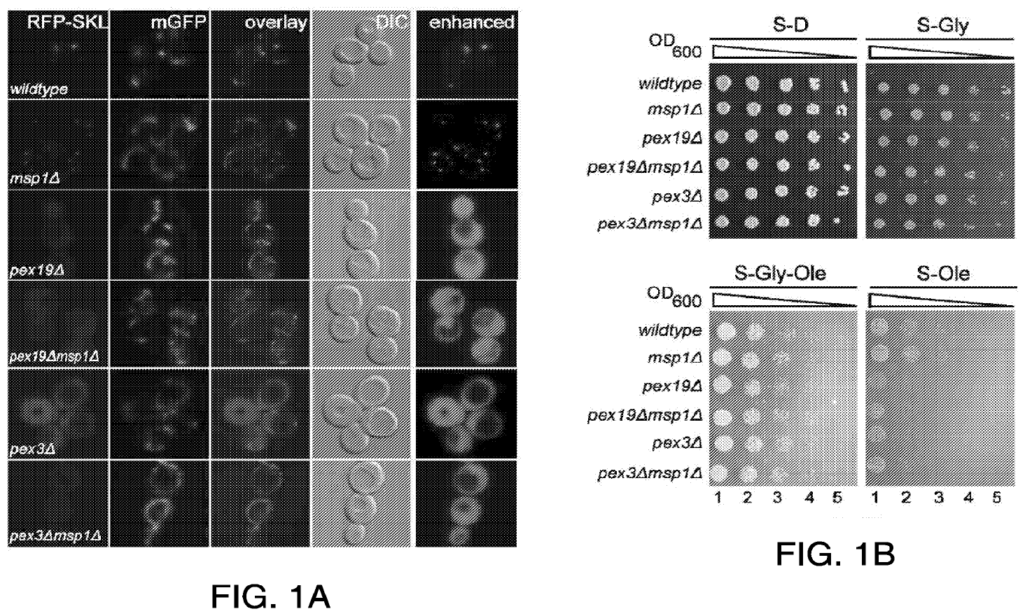

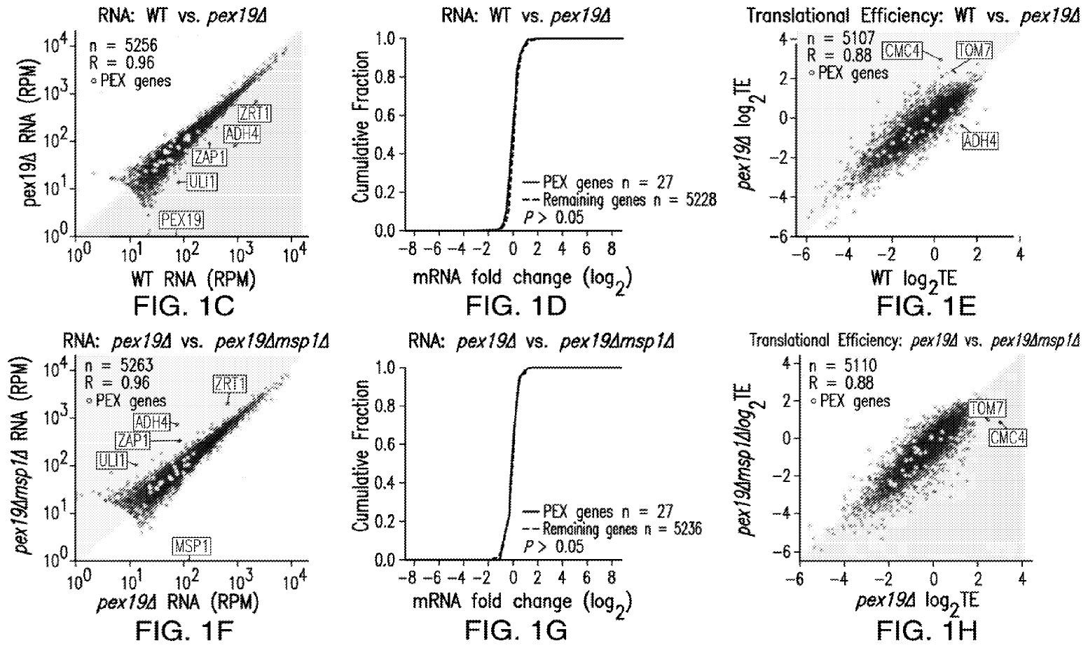

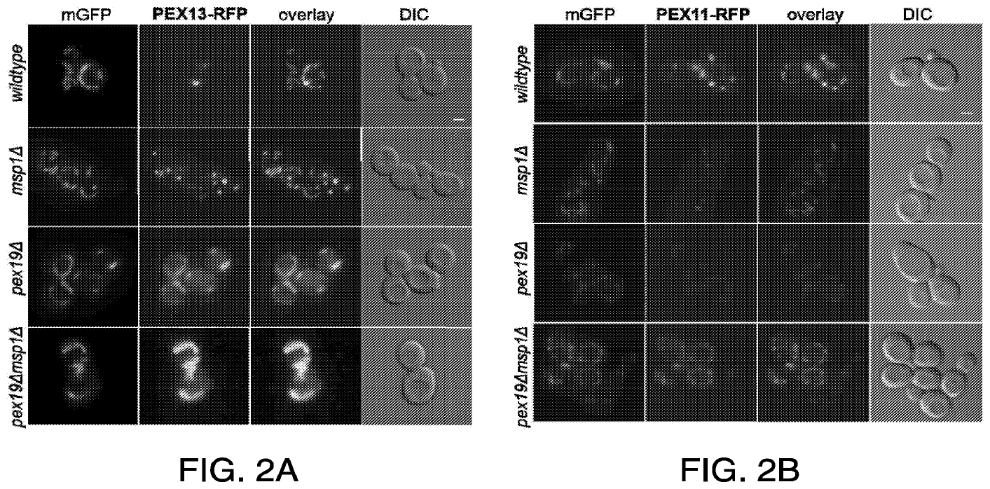

Results. Peroxin gene expression is maintained in the absence of peroxisomes. Experiments were carried out to globally define the fate of peroxisomal proteins and the regulation of peroxisomal genes in PBDs. Using the budding yeast S. cerevisiae as a model system, pex3 and pex19, which have been previously shown to cause loss of peroxisomes, were depleted. As mentioned earlier, some peroxisomal proteins such as Pex15 can become mis-localized to the mitochondria, especially when quality control proteins like Msp1 known to be beneficial in the context of mitochondrial protein removal are disturbed and help to exacerbate resulting phenotypes. Therefore, single and double deletions of pex3 and msp1 as well as pex19 and msp1 were generated. The presence of peroxisomes was monitored by expressing fluorescent proteins fused to the peroxisomal targeting motif SKL which labeled the punctate peroxisomes present in wild-type and msp1l cells (FIG. 1A). As expected, in pex3Δ and pex19Δ cells, as well as in pex3Δmsp1Δ and pex19Δmsp1Δ punctate peroxisomes were not detected. Instead, faint fluorescence was visible throughout the cytosol, which suggests the absence of intact, functional peroxisomes which would normally import the fluorescent protein (FIG. 1A). To confirm that there is no remaining peroxisomal function despite the loss of punctate peroxisomes, the strains were plated on oleate-containing media. To metabolize oleate, yeast requires functional peroxisomes otherwise very long chain fatty acids become toxic to the cell when offered as the carbon source. Both the pex3Δ and pex194 deletion strains as well as pex3Δmsp1Δ and pex19Δmsp1Δ do not have a phenotype on glucose or glycerol (FIG. 1B, upper panels), but have a clear growth defect relative to wild-type on oleate (FIG. 1B, lower panels). These results demonstrate loss of peroxisomes and ablation of peroxisome-mediated metabolism in pex3Δ and pex19Δ and pex3Δmsp1Δ and pex19Δmsp1Δ yeast.

Next, experiments were carried out to understand the regulation of peroxisomal genes in yeast that lack peroxisomes. It is unknown if there exists any quality control mechanism in yeast to downregulate expression of peroxisomal genes in the absence of functional peroxisomes. In addition, translation of some nuclear-encoded mitochondrial proteins is coupled to their import into the organelle; imported peroxisomal proteins could similarly be regulated at the level of translational efficiency (TE). Therefore, ribosome-footprint profiling was performed to measure differences in both gene expression and TE between wild-type and peroxisome-deficient pex19Δ yeast. Despite lacking a highly conserved organelle, global gene expression is remarkably similar in wild type and pex19Δ yeast (FIG. 1C). More importantly, transcription of peroxisomal proteins is also not affected (FIG. 1D). A similar pattern is observed for TE as well (FIG. 1E). Deletion of Msp1 alone or in the pex194 background has no apparent effect on peroxin gene expression or TE, as expected given its post-translational role in protein abundance and localization (FIGS. 1F, 1G, 1H). However, three zinc-response genes (ADH4, ZAP1, and ZRT1) that are downregulated in pex19Δ yeast relative to wild-type and msp1Δpex191 (FIGS. 1C, 1F) were detected. This set includes ZAP1, which is the principle transcription factor and zinc sensor that coordinates zinc homeostasis and ZRT1 a zinc transporter indicating that there is a peroxin independent response. Overall, these experiments indicate that the absence of peroxisomes does not significantly impact the expression or translation of peroxin-encoding genes.

Peroxisomal proteins accumulate on mitochondria in absence of peroxisomes in yeast. The destiny of peroxins was explored on the protein level since the transcription and translation of peroxin RNA levels and their translational efficiency (TE) is maintained.

The data obtained with ribosome profiling indicate that peroxisomal proteins are still translated and search the cell for their destination. Therefore, it was tested whether proteins that normally localize to peroxisomes are now getting rerouted to another destination. As the peroxisomal protein Pex15 is one of the mitochondrially mislocalized proteins in get1 and get3 mutants, Pex- and Msp1-single and double deletion strains were used.

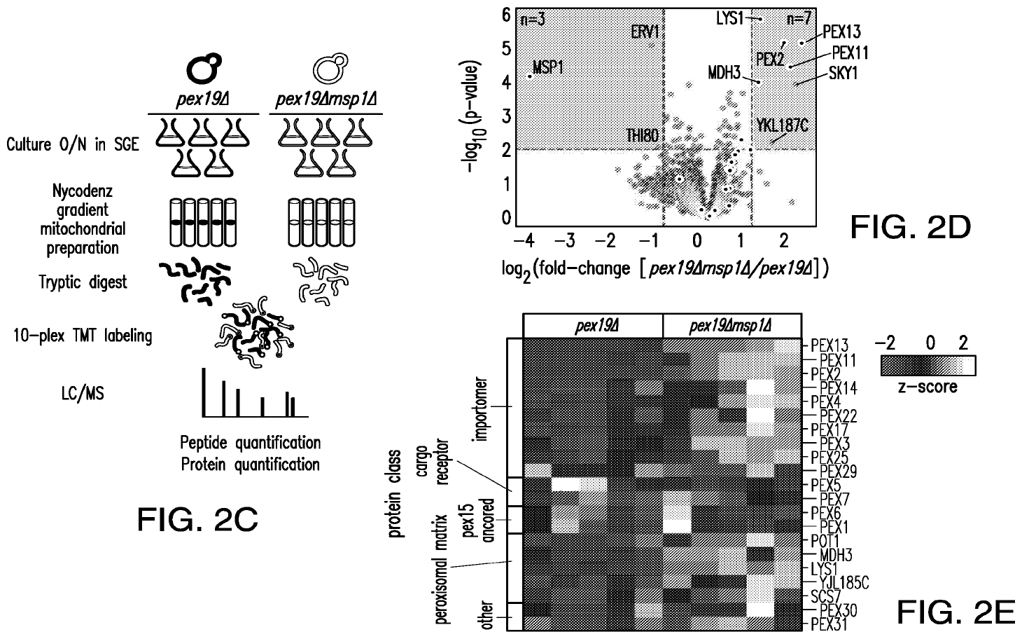

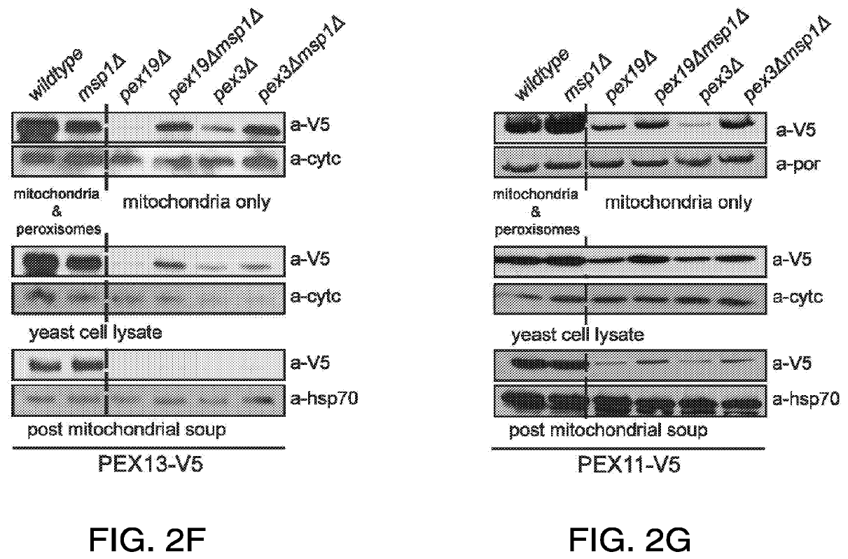

To test whether peroxisomal proteins might get rerouted to mitochondria, some example peroxins were first visualized in the cell. Two peroxins, Pex13 and Pex11, were fused with RFP and the yeast cells were imaged. Indeed, the RFP signal that was observed was completely overlaying with the mito-GFP signal in both strains, the single deletion (pex19Δ) as well as in the double deletion (pex19Δmsp1Δ) (FIGS. 2A, 2B). This finding indicates that as soon as peroxisomes are absent the peroxins, they find the mitochondria as their new destination. Even though statistically not significant, in some images of the double deletion strain (pex19Δmsp1Δ) increasing fluorescence was detected indicating protein accumulation in the absence of Msp1 as expected. With this evidence, it was likely that a mass spectrometry analysis would reveal the peroxisomal proteome which reroutes to mitochondria. Additionally, quantitative mass spectrometry will provide quantitative insight about the peroxin accumulation in the double deletion (pex19Δmsp1Δ) versus the single deletion (pex19Δ).

To investigate this question, mitochondria were isolated from the strains missing peroxisomes (pex19Δ and pex19Δmsp1Δ) using a nycodenz gradient to perform quantitative mass spectrometry. It is known that peroxisomes and mitochondria form contact sites similar to those between the ER and mitochondria as well as that they do cofractionate. Therefore, it was assumed that there will be contamination even when using a nycodenz purified mitochondrial fraction (as seen in FIGS. 2F, 2G), so strains which still have peroxisomes (wild-type and msp1Δ) were excluded. Detecting peroxisomal proteins in the mitochondrial fraction of the deletion strains (pex19Δ, pex19Δmsp1Δ) on the other hand completely excludes the possibility of peroxisomal contamination and therefore will give interpretable results about the presence of peroxisomal proteins on and within mitochondria. The proteome of the selected strains pex19Δ versus pex19Δmsp1Δ were analyzed using quantitative tandem mass spectrometry after isobaric labeling. Briefly, the mitochondrial fractions are normalized to protein level and peptides are covalently labeled with stable isotope molecules with tags of varying mass introduced from TMT reagents of peptides after proteolysis (FIG. 2C). The analysis of the mitochondrial proteome revealed increased abundance of 15 peroxisomal proteins within the mitochondrial fraction of pex19msp1Δ yeast cells (FIGS. 2D, 2E). The top candidates of the increased proteins included peroxisomal proteins such as Pex11, Lys1, Pex2, Pex13, and Mdh3. Pex2 and Pex13 that belong to the class of peroxins; Mdh3 is the peroxisomal malate dehydrogenase and Lys1 is involved in lysine biosynthesis and is localized to the cytosol as well as the peroxisome. It was concluded that peroxisomal proteins are still synthesized and some of them belong now to the mitochondrial proteome (FIGS. 2D, 2E). The discovery of Pex13 and Pex11 as the most enriched proteins led to the exploration of this class of proteins in more detail. The setup of the experiment which focused on strains not having peroxisomes revealed the peroxisomal proteins which belong to the mitochondrial proteome under those circumstances. The peroxins Pex13, 11, 2, 14, 4, 22, 17, 3 and 25 as well as 3 peroxisomal matrix proteins (Pot1, Mdh3, Atg36) (FIG. 2E) were identified. It was also found that Scs7 which is known to participate in alpha-oxidation, a peroxisomal exclusive pathway, which is so far not annotated for a specific organelle (FIG. 2E). Some components of the peroxisomal import machinery do not accumulate (Pex 5, 7, 1, 6, and 30) in mitochondria (FIG. 2E). Pex5 and Pex7 are the peroxins which shuttle the cytosol to find cargo proteins and integrate in the membrane for a short amount of time to release their cargo. Pex1 and Pex6 themselves are not embedded into the membrane and use a membrane anchor.

To exclude that this result is pex19Δ relevant, we did the same experiment with pex3Δ versus the double deletion pex3Δmsp1Δ and achieved similar results.

Peroxisomal import is facilitated by the peroxisomal importomer which is a transient multi protein assembly. Briefly, receptors recognize their cargo in the cytosol. They shuttle their cargo to the peroxisome where binding to the docking complex (Pex13,14,17) occurs. Involvement of the translocon formed by RING-finger peroxins (Pex2,10,12) followed by ubiquitination (Pex4, Pex22) and therefore recycling of the receptor by the AAA-ATPase complex formed by Pex1 and 6 is finalizing the process. Interestingly, most of the peroxins which were detected in mitochondria represent members of the peroxisomal import machinery, called importomer.

To validate the apparent hits of the quantitative mass spectrometry, Pex13, the most enriched peroxin, as well as Pex11 was V5-tagged and transformed the construct into the wild-type, msp1Δ, pex19Δ, and pex19Δmsp1Δ1 strains and isolated mitochondria. Again, the mitochondrial fraction was purified with a nycodenz gradient to isolate intact pure mitochondria. As expected, the Western blot signal in strains containing mitochondria and peroxisomes (wild-type, msp1Δ) showed a strong signal for the V5-tagged proteins (FIG. 2 F, 2G), which is due to peroxisomal contamination caused by co-purification (confirmed by the detection of peroxisomal catalase, an enzyme which is detected in fully assembled mature peroxisomes in the wild-type samples. On the other hand, in peroxisomal deletion strains, a faint band was detected in pex19Δ single for Pex13-V5 and Pex11-V5, and the enrichment of the individual peroxins in the double deletion strain was confirmed (FIGS. 2F, 2G). The low abundant signal in the single deletion was expected according to the quantitative mass spectrometry results and microscopy data. This experiment was also performed with one peroxin which did not accumulate on the mitochondria, Pex30, according to the MS experiment. Pex30-V5 did not accumulate on mitochondria, which confirms the specificity of i) the used mass spec technique, and ii) the specific subset of accumulating peroxins and peroxisomal matrix proteins on a biological level.

Described herein is an extensive profile of peroxins and other proteins that are accumulating in the mitochondrial membrane especially when the mitochondrial quality control is impaired.

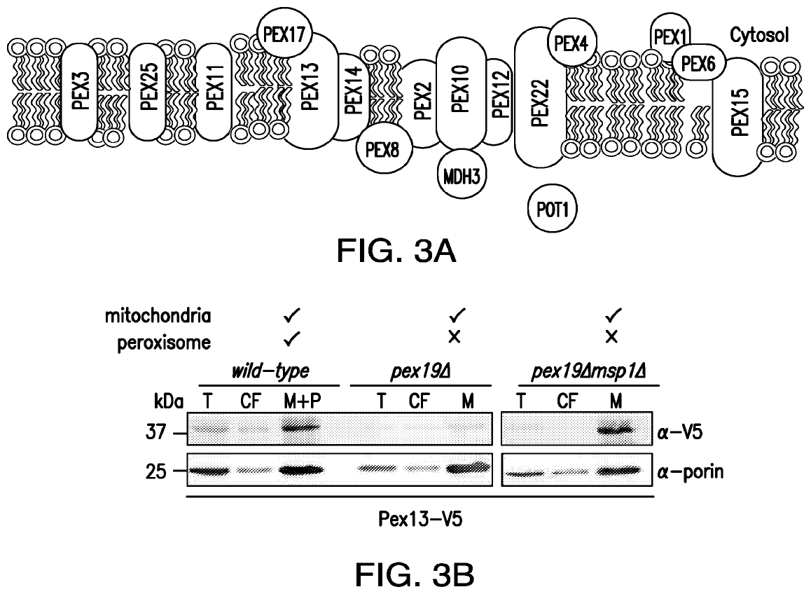

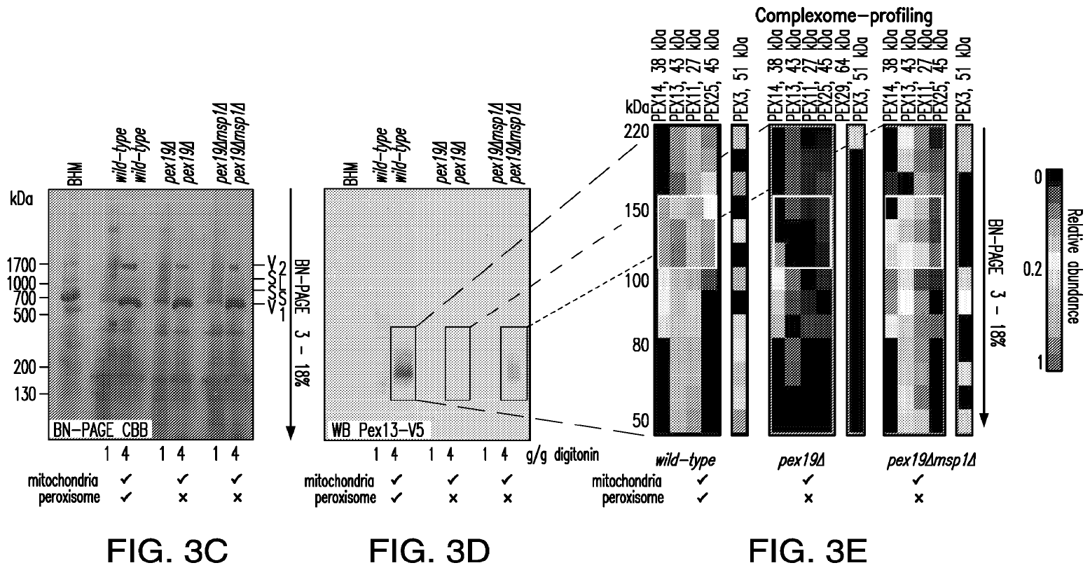

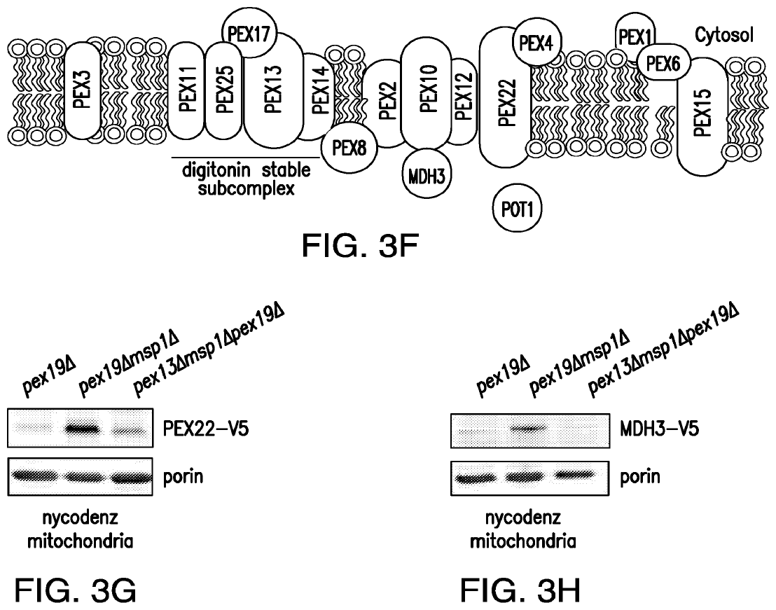

BN-PAGE of yeast mitochondria reveals a high molecular weight assembly of peroxins on mitochondria. Some peroxisomal matrix proteins were detected that have never been reported to be localized to the mitochondria before (FIG. 2 and FIG. 3A (summary cartoon)). Therefore, it was tested whether the enrichment of peroxins on mitochondria is a random accumulation or a structured event. To address this question, the yeast strains were transformed with Pex13-V5, isolated mitochondria (FIG. 3B) and separated on a 3-18% gradient Blue Native PAGE using different amounts of detergent (FIG. 3C). Given that it is being investigated if the peroxins are in contact with each other in the mitochondrial membrane which uses 3 to 6 g digitonin per g mitochondrial membrane, stability versus resolution was assessed with different amounts of detergent. The extensively studied bovine heart mitochondria was used as an internal molecular weight marker on the gel. With one g/g detergent to membrane ratio, the mitochondrial membrane to smear was detected over the gel, but the V5 signal of Pex13-V5 started at around 1000 kDa in size (FIGS. 3C and D lanes marked with 1 g/g). When increasing the amount of digitonin, higher molecular weight assemblies were disrupted but a complex of 180-200 kDa in size (FIGS. 3C, 3D; lanes marked with 4 g/g) was retained. In organisms with a peroxisome, Pex13 forms a complex with Pex14, Pex17 and putatively Pex8 with a total mass of 172.5 kDa. Therefore, the detection of a complex based on the V5 tagged Pex13 of about 150-180 kDa in size predicted that Pex13 might interact with some of its native interaction partners (FIG. 3D). This interaction is expected in the wild-type strain (FIGS. 3C, D and E) but we could also detect a very faint assembly in the pex19Δ strain and with even stronger signal in the pex19Δmsp1Δ strain. We conclude that in the absence of peroxisomes the docking complex of the peroxisomal import machinery is assembling on the mitochondria. (FIGS. 3A, D, and E). To further investigate the composition of the subcomplex, detection at 150 to 180 kDa (FIG. 3D) and separation of the mitochondrial membranes with a detergent ratio (3 g/g) in between the two previously tested ones was done to establish the bliss point between disassembly and resolution (FIG. 3E). With this method 13 peroxins were detected, and the abundance was followed through the different molecular weight ranges of the peroxins forming the docking complex, (Pex13, Pex14 and Pex17). It was found that Pex13, Pex14 as well as Pex11, and Pex25 are component s of the detected subcomplex. Taken together, the accumulation of Pex11 and Pex25 was identified with the known components of the traditional importomer (Pex13 and Pex14) with quantitative mass spectrometry (FIG. 2) and complexome-profiling (FIG. 3). It was confirmed that Pex13 started to appear at a molecular weight of 400 kDa in the wild-type with its highest abundance at around 180 kDa. In the pex19Δ single deletion, this pattern was faint, but in the double deletion pex19Δmsp1Δ, the described pattern reappeared. Pex14 had its highest abundance between 170 to 100 kDa. Interestingly, the pattern matches that of the wild-type providing evidence that the native interaction of Pex13 and 14 is reoccurring at the mitochondrial membrane (FIG. 3D, white box). If the proteins were monomeric and randomly sticking in the membrane, a single peak of that peroxin at the running front in the BN-PAGE would be expected. This seems to hold true for Pex17, even though the results show it appearing at around 300 kDa and at the running front. This could either be an indication for oligomerization or that Pex17 is partially associated with Pex13 and Pex14, but since it is known to interact with Pex14 on the cytosolic side without being embedded into the membrane, this pattern might be reflective of detergent sensitivity. Also, the function of Pex17 and how it is contributing to the docking mechanism is still elusive, since this peroxin is not conserved in mammals. The shown complexome dataset reveals a subcomplex composed of Pex13, 14, 11 and 25 which appears at 150 to 180 kDa in size (FIG. 3D, white box). These components were reported to belong to the importomer. Calculating the molecular weight of this complex based on the individual masses of the components would result in 153 kDa and therefore matches the detected band in the BN-PAGE (FIG. 3D) as well as in the complexome profile (FIG. 3E). Therefore, the results show that in the absence of peroxisomes, a digitonin stable subcomplex and putatively the entire importomer assembles in the mitochondrial membrane (FIG. 3E).

The role of PEX13 as part of the docking complex when localized to mitochondria. Pex13 is part of the importomer and could be the initiator of docking target proteins for translocation. Pex13 might also be the signaling molecule on the specialized endoplasmic reticulum giving rise to preperoxisomal vesicles.

Taken the data together, it was evaluated whether Pex13 might fulfill a significant signaling role as soon it is embedded into the outer mitochondrial membrane by putatively attracting other peroxins as well as peroxisomal matrix proteins to the organelle. Therefore, new deletion strains were created that lack Pex13 in the background of pex19Δ, msp1Δ, and pex19msp1Δ. If the loss of Pex13 abolishes the subcomplex formation on mitochondria, the loss of the accumulation of the other peroxins would be expected. To this end, newly generated deletion strains were transformed with Pex22-V5, isolating mitochondria and detection of the V5-tag. The results demonstrate that the Pex22-V5 signal is significantly reduced in the triple deletion strain (FIG. 3F). The formation of a functional docking complex which allows accumulation of other peroxins would also explain why specific peroxisomal matrix proteins are localizing to the mitochondria (FIGS. 2D, 2E). Therefore, the strains (pex19Δ, pex19Δmsp1Δ and pex13Δpex19Δmsp1Δ were transformed with Mdh3-V5 and its abundance on mitochondria was traced. Again, the results confirm that Mdh3 was accumulating in a Pex13 dependent manner. Docking complex formation therefore is an entry route for peroxisomal proteins into the mitochondria. These results, taken together, can explain how the mitochondria malfunctions. The results also show that the subcomplex found on mitochondria is a functional unit which supports the accumulation of peroxins and peroxisomal matrix protein in mitochondria and establishes Pex13 as putative important signaling protein initiating formation of this importomer.

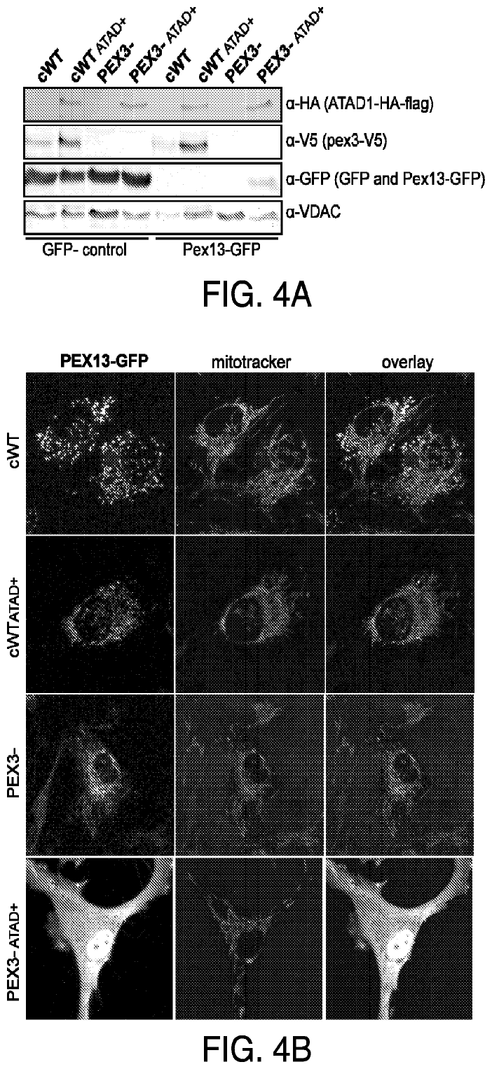

Peroxins target to mitochondria in human Zellweger Syndrome fibroblasts. Next, it was tested whether peroxin accumulation is also conserved in humans. A PEX3 deficient (PEX3⁻) fibroblast cell line derived from a patient with Zellweger Syndrome was used, and a complemented wild-type cell line was created by re-expressing PEX3 (cWT) using lentivirus (FIG. 4B). This restores the cells phenotype as well as the peroxisomal structures. ATAD1 was overexpressed in both cell lines (cWT^{ATAD+}, PEX3^{-ATAD+}) (FIG. 4A), which results in a total of 4 cell lines (cWT, cWT^{ATAD+}, PEX3⁻, PEX3^{-ATAD+}) to test ATAD1's role in removing accumulating proteins.

First, it was examined whether peroxins are localizing to mitochondria. PEX13 was cloned into a lentiviral construct and fused with GFP. The transfected cell lines were enriched for PEX13-GFP positive cells by flow cytometry and subjected to live cell fluorescent imaging. A GFP-signal was detected in cWT, and cWT^{ATAD+} cells displaying a punctate pattern. In PEX3⁻ cells, the PEX13-GFP signal was dim but overlaying with the mitochondrial network entirely, while in PEX3^{-ATAD+} cells, a bright signal was exerted similar to the free GFP transfection control. To visualize the mitochondrial network, Mitotracker far red was used (FIG. 4B). The GFP-signal in the cytosol and nucleus indicates that PEX13-GFP is not stabilized in the mitochondrial outer membrane (FIG. 4B) This suggests that ATAD1 is able to remove peroxisomal proteins from the mitochondrial membrane. Since it is unlikely to believe that a membrane protein can be evenly distributed throughout the cytosol, it was confirmed that the bright green signal was free GFP which was proteasome stable, while the full-length protein is degraded (FIG. 4A). The full length PEX13-GFP signal was detectable with imaging the Western blot membrane overly saturated, in line with the observation that expression of PEX13-GFP results in very low levels of detectable green fluorescence. To confirm this finding, PEX12-GFP was transiently expressed in 4 cell lines (cWT, cWT^{ATAD+}, PEX3⁻, PEX3^{-ATAD+}) and a very similar distribution with a slightly milder effect in the ATAD1 overexpressing patient cell line (PEX3⁻) was detected. This assay was also performed in another Zellweger Syndrome patient cell line with a pex16Δ null deletion chasing PEX11-GFP (ref. https://collections.lib.utah.edu/ark:/87278/s6zd2mph). The result is similar to the result described here, and therefore excludes the possibility of monitoring a specific phenotype of a PEX3 null deletion.

It was confirmed that peroxins are localizing to mitochondria in Zellweger Syndrome. Additionally, it was also confirmed that ATAD1 is involved in the removal of these peroxins from the outer mitochondrial membrane, similarly to its function in removing tail anchored proteins in GET system impaired cells. Because of the low expression level of human PEX13-GFP, yeast mitochondria were used and a pulldown from MSP1-E198Q was performed showing that a faint signal was detected indicating a putative interaction of MSP1 with PEX13.

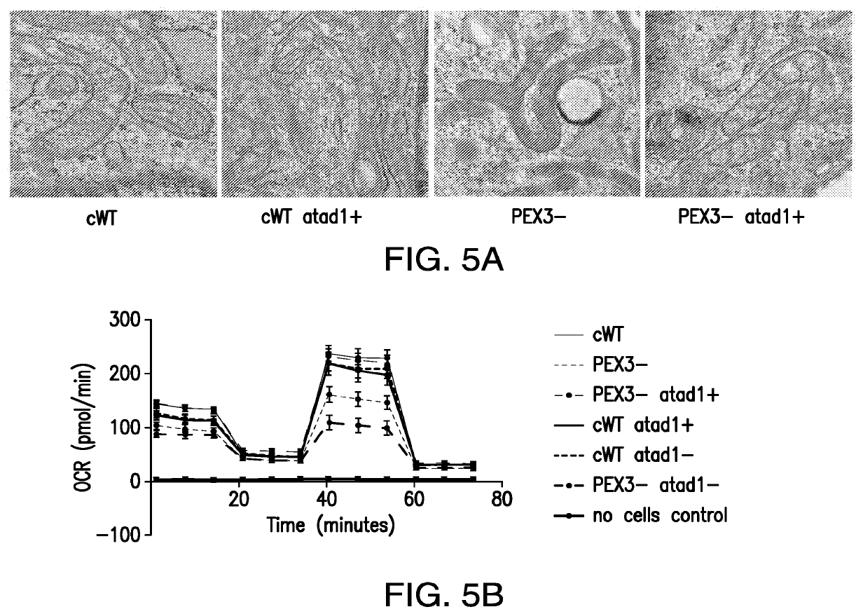

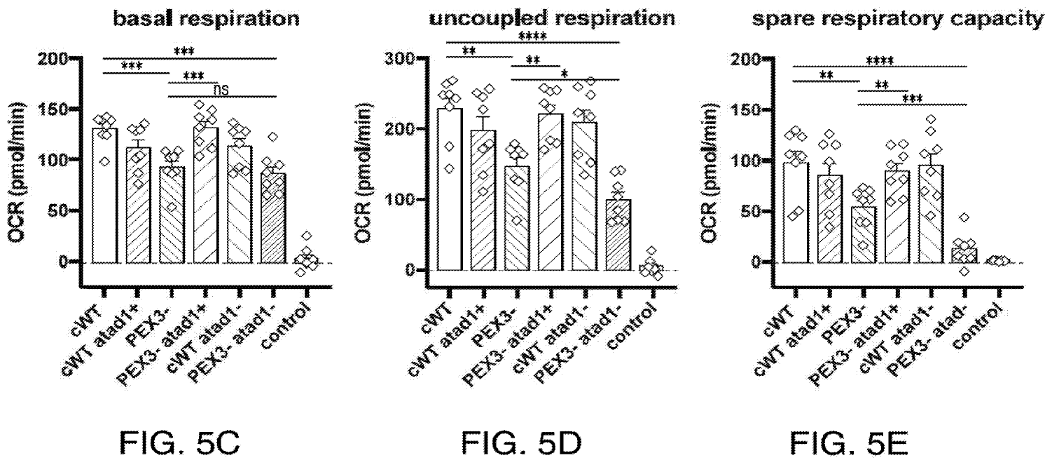

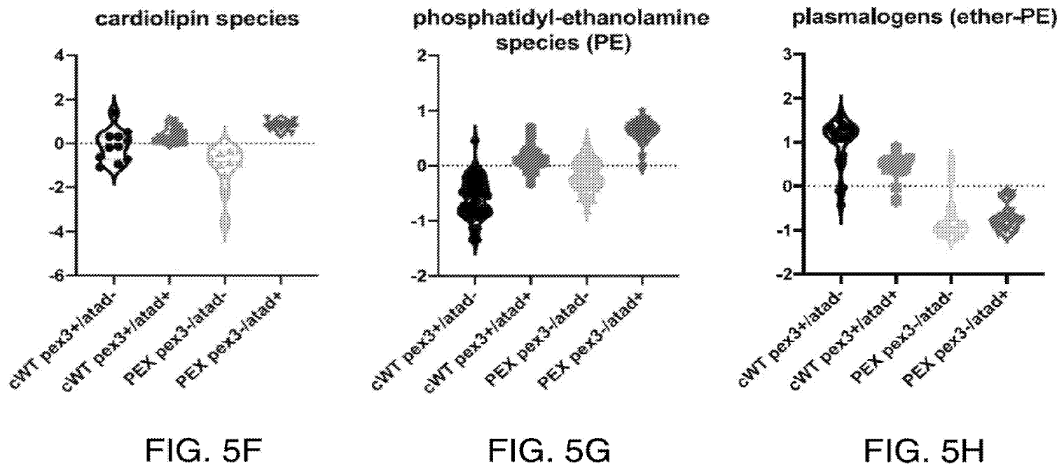

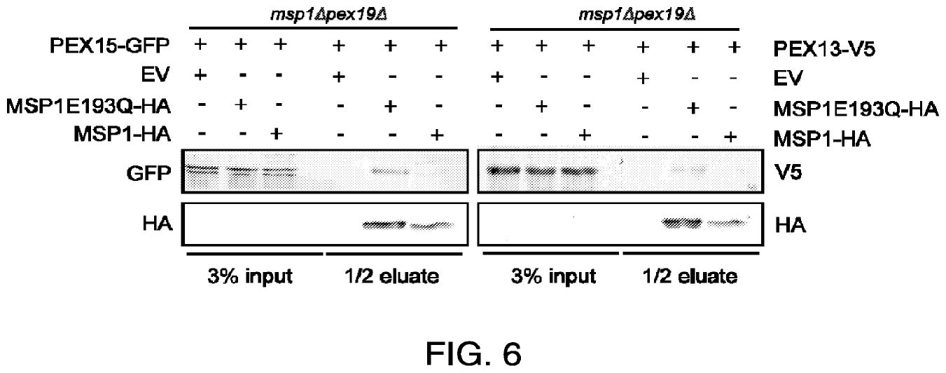

Over expression of ATAD1 rescues the mitochondrial phenotype in human PBD/ZSD fibroblasts. Based on the findings described herein in yeast, it was tested whether peroxin mis-localization causes a phenotype and if ATAD1 overexpression would rescue it. First, mitochondrial morphology was investigated. Indeed, morphology was altered as shown in representative images of electron microscopy (EM) (FIG. 5A; cWT versus PEX3⁻). Cristae are barely detectable in the patient cell line and it becomes apparent that the electron density in the patient mitochondria is higher compared to wild-type. (FIG. 5A). Electron density is caused by osmium stain which is achieved when osmium is reacting with protein. Interestingly, in the patient cell line overexpressing ATAD1, the electron density recovers and cristae formation is partially detected. In images of wild-type cells, some variation of mitochondrial morphology was observed. Three categories of mitochondria were classified: mitochondria with cristae and low electron density (type 1), mitochondria with fewer cristae (type 3) and damaged mitochondria with high electron density and/or almost no cristae (type 2). Damaged mitochondria with different morphologies was observed. Interestingly, more than 80% of the mitochondria in the patient was accounted as type 2, whereas in the wild-type cells about 80% of the mitochondria was accounted as types 1 and 3. When categorizing the PEX3^{-ATAD+} cells, we found that the morphology phenotype could be reversed and about 70% of cells were accounted as type 3.

Based on the observed morphology, the results showed that the overexpression of ATAD1 rescues mitochondrial morphology suggesting it might also improve respiration. Therefore, it was tested whether removal of the accumulated peroxins is able to restore mitochondrial respiration. To test the direct dependence of this phenotype with the expression levels of ATAD1, additional cell lines with genetic deletion of ATAD1 was generated. A Seahorse XF 96e Flux analyzer was used to measure respiration (FIG. 5B). A significant lower basal rate of respiration in the PEX3⁻ cell line was observed compared to the complemented wild-type cWT (FIGS. 5B, 5C). Also, the response to the uncoupler FCCP was significantly lower in the patient cell line (PEX3⁻) compared to wild-type (cWT) (FIG. 5B,D), indicative of lower respiratory capacity of those cells (FIG. 5E). Surprisingly, when ATAD1 was overexpressed in the patient cell line (PEX3^{-ATAD+}), basal mitochondrial respiration was restored back to wild-type level (FIGS. 5B, 5C). The uncoupled respiration normalized to wild-type respiration (FIGS. 5B, 5D) and therefore recovered the mitochondrial spare capacity (FIG. 5E). In the case that the expression level of ATAD1 is sufficient to cause the phenotype, it would be expected that a much more severe phenotype in the patient with an additional ATAD1 deletion. By measuring an even lower basal and uncoupled respiration, this expectation was confirmed along and its impact on the mitochondrial spare capacity in the PEX3^{-ATAD1-} cell line (FIGS. 5B, 5C, 5D, 5E).

To complement the characterization with other readouts relevant to reflect important mitochondrial function, unbiased whole cell lipidomics was performed. Interestingly, the lipid class of cardiolipins is changing to lower levels in the patient cell lines but normalizes to wild-type levels with the overexpression of ATAD1 (FIG. 5F). Another readout of flourishing mitochondrial metabolism is the capacity of mitochondria to import decarboxylase phosphatidylserines (PSs) into phosphatidylethanolamines (PEs). This species is detected with slightly higher values than in wild-type (FIG. 5G). The class of phospho-ether lipids (plasmalogens), precursors to myelin, seems to be unaffected (FIG. 5H). This does not come by surprise since the relevant enzymes are solely active and present when intact and mature peroxisomes are built.

As described herein, the results demonstrate that mitochondrial respiration can be rescued in a Zellweger patient cell line by overexpression of ATAD1. ATAD1 is important and sufficient to impact mitochondrial respiration in Zellweger Syndrome patient cell lines. Furthermore, the lipid classes indicative of mitochondrial health normalized (FIGS. 5F, 5G). The mitochondria seemed less condensed, less electron dense and cristae formation was detectable (FIG. 5A). For the first time, the mitochondrial phenotype in Zellweger Syndrome, which is contributing to the severe neuronal symptoms, can be rescued.

FIGS. 5A-G shows that ATAD1 rescues the phenotype in human ZS fibroblasts.

What is claimed is:

1. A composition comprising a peptide, wherein the peptide comprises the sequence of SEQ ID NO: 1, or a variant or a fragment thereof.

2. The composition of claim 1, wherein the fragment of SEQ ID NO: 1 has an amino acid sequence of at least 90% sequence identity to SEQ ID NO: 1.

3. The composition of claim 1, wherein the variant of SEQ ID NO: 1 has an amino acid sequence of at least 90% sequence identity when compared to SEQ ID NO: 1.

4. The composition of claim 1, wherein the peptide is capable of binding one or more peroxins.

5. The composition of claim 1, wherein the peptide is capable of recognizing, binding and removing one or more peroxins embedded in an outer mitochondrial membrane of a cell.

6. The composition of claim 4, wherein the one or more peroxins is a tail-anchored protein.

7. The composition of claim 4, wherein the one or more peroxins is not a tail-anchored protein.

8. The composition of claim 4, wherein the one or more peroxins is PEX13, PEX11, PEX2, PEX17, PEX3, PEX22, PEX25, PEX14, PEX4 or a combination thereof.

9. The composition of claim 1, further comprising a therapeutic agent, wherein the therapeutic agent is baclofen or keppra.

10. A pharmaceutical composition comprising a peptide, wherein the peptide comprises the sequence of SEQ ID NO: 1, or a variant or a fragment thereof and a pharmaceutically acceptable carrier.

11. A composition comprising a nucleic acid capable of encoding a peptide comprising the sequence of SEQ ID NO: 1, or a fragment or a variant thereof.

12. A vector comprising the nucleic acid of claim 11.

13. A pharmaceutical composition comprising the vector of claim 12.

14. The pharmaceutical composition of claim 13, further comprising a pharmaceutically acceptable carrier.

15. The pharmaceutical composition of claim 13, wherein the pharmaceutically acceptable carrier comprises a lipid-based or polymer-based colloid.

16. The pharmaceutical composition of claim 15, wherein the colloid is a liposome, a hydrogel, a microparticle, a nanoparticle or a micelle.

17. The pharmaceutical composition of claim 10 or 13, wherein the pharmaceutical composition is formulated for intravenous, oral, intramuscular, intraperitoneal, subcutaneous or intrathecal administration.

18. A method of removing one or more peroxins embedded in the outer mitochondrial membrane of a cell in a subject, the method comprising: administering a therapeutically effective amount of the composition of claim 1 or the pharmaceutical composition of claim 10 or claim 13, wherein the therapeutically effective amount of the composition or pharmaceutical composition removes one or more peroxins embedded in the outer mitochondrial membrane.

19. A method of reducing peroxin accumulation in the outer mitochondrial membrane of a cell in a subject, the method comprising: administering a therapeutically effective amount of the composition of claim 1 or the pharmaceutical composition of claim 10 or claim 13, wherein the therapeutically effective amount of the composition or pharmaceutical composition reduces peroxin accumulation in the outer mitochondrial membrane of a cell in the subject.

20. The method of claim 19, wherein the therapeutically effective amount of the composition of claim 1 or the pharmaceutical composition of claim 10 or claim 13 removes one or more peroxins embedded in the outer mitochondrial membrane of a cell in the subject.

21. A method of rescuing mitochondrial function in a cell of a subject, the method comprising: administering a therapeutically effective amount of the composition of claim 1 or the pharmaceutical composition of claim 10 or claim 13, wherein the therapeutically effective amount of the composition or pharmaceutical composition rescues mitochondrial function in the cell of the subject.

22. The method of claim 21, wherein the therapeutically effective amount of the composition of claim 1 or the pharmaceutical composition of claim 10 or claim 13 removes one or more peroxins embedded in the outer mitochondrial membrane of a cell in the subject.

23. A method of treating a subject that has or has been diagnosed with a perioxisomal biogenesis disorder, the method comprising: administering a therapeutically effective amount of the composition of claim 1 or the pharmaceutical composition of claim 10 or claim 13, wherein the therapeutically effective amount of the composition or pharmaceutical composition removes one or more peroxins embedded in the outer mitochondrial membrane of a cell in the subject, reduces peroxin accumulation in the outer mitochondrial membrane of a cell in the subject, or rescues mitochondrial function in a cell of the subject, thereby treating the subject.

24. A method of reducing one or more symptoms of a perioxisomal biogenesis disorder in a subject, the method comprising: administering a therapeutically effective amount of the composition of claim 1 or the pharmaceutical composition of claim 10 or claim 13, wherein the therapeutically effective amount of the composition or pharmaceutical composition removes one or more peroxins embedded in the outer mitochondrial membrane of a cell in the subject, reduces peroxin accumulation in the outer mitochondrial membrane of a cell in the subject, or rescues mitochondrial function in a cell of the subject, thereby reducing one or more symptoms of a perioxisomal biogenesis disorder in the subject.

25. The method of claim 24, wherein the one or more symptoms are neonatal seizures, hepatomegaly, renal cysts, skeletal abnormalities, impaired hearing, weakness, poor eyesight and shortness of breath.

26. A method of treating a subject with a deficiency in human ATAD1 levels or expression, the method comprising: administering a therapeutically effective amount of the composition of claim 1 or the pharmaceutical composition of claim 10 or claim 13, wherein the therapeutically effective amount of the composition or pharmaceutical composition removes one or more peroxins embedded in the outer mitochondrial membrane of a cell in the subject, reduces peroxin accumulation in the outer mitochondrial membrane of a cell in the subject, or rescues mitochondrial function in a cell of the subject, thereby treating the subject.

27. A method of increasing human ATAD1 protein or gene levels or expression in a cell in a subject, the method comprising: contacting a cell with a therapeutically effective amount of composition of claim 1 or the pharmaceutical composition of claim 10 or claim 13, wherein the therapeutically effective amount of the composition or pharmaceutical composition removes one or more peroxins embedded in the outer mitochondrial membrane of a cell in the subject, reduces peroxin accumulation in the outer mitochondrial membrane of a cell in the subject, or rescues mitochondrial function in the cell in the subject.

28. The method of any of claims 18-27, wherein the therapeutically effective amount of the composition or pharmaceutical composition is administered orally, intramuscularly, intraperitonealy, intravenously, subcutaneously or intrathecally.

29. The method of any of claims 18-27, wherein the subject has a perioxisomal biogenesis disorder.

30. The method of claim 29, wherein the perioxisomal biogenesis disorder is Zellweger Syndrome, neonatal adrenoleukodystrophy, or infantile Refsum disease.

31. The method of claim 30, wherein the subject has Alzheimer's disease, Parkinson's disease, multiple sclerosis, or an age-related disease.

32. The method of any of claims 18-31, wherein the subject is a human.

33. The method of any of claims 18-31, wherein the one or more peroxins embedded in the outer mitochondrial membrane are PEX13, PEX11, PEX2, PEX17, PEX3, PEX22, PEX25, PEX14, PEX4 or a combination thereof.

34. The method of any of claims 18-32, further comprising administering baclofen or keppra to the subject.

35. A method of restoring mitochondrial respiration in a subject, the method comprising: administering a therapeutically effective amount of the composition of claim 1 or the pharmaceutical composition of claim 10 or claim 13, wherein the therapeutically effective amount of the composition or pharmaceutical composition restores mitochondrial respiration in the subject.

36. A method of restoring mitochondrial respiration in a cell, the method comprising: contacting a cell with a therapeutically effective amount of the composition of claim 1 or the pharmaceutical composition of claim 10 or claim 13, wherein the therapeutically effective amount of the composition or pharmaceutical composition removes one or more peroxins embedded in the outer mitochondrial membrane of a cell in a subject, reduces peroxin accumulation in the outer mitochondrial membrane of a cell in a subject, or rescues mitochondrial function in a cell of a subject.

37. A method of treating a deficiency in human ATAD1 levels or expression in a cell, the method comprising: contacting a human cell with a therapeutically effective amount of the composition of claim 1 or the pharmaceutical composition of claim 10 or claim 13, wherein the therapeutically effective amount of the composition or pharmaceutical composition removes one or more peroxins embedded in the outer mitochondrial membrane of a cell in the subject, reduces peroxin accumulation in the outer mitochondrial membrane of a cell in the subject, or rescues mitochondrial function in a cell of the subject.

38. A method of inhibiting the import of one or more peroxisomal matrix proteins into the mitochondria of a cell, the method comprising: contacting the cell with a therapeutically effective amount of the composition of claim 1 or the pharmaceutical composition of claim 10 or claim 13, wherein the peptide of the composition or pharmaceutical composition is capable of binding to the peroxisomal matrix protein.

39. The method of claim 38, wherein the one or more peroxisomal matrix proteins are POT1, MDH3, or ATG36.


SEQUENCE LISTING (2 sequences)

SEQ ID NO: 1 - protein, 361 aa, Homo sapiens
MVHAEAFSRPLSRNEVVGLIFRLTIFGAVTYFTIKWMVDAIDPTRKQKVEAQKQAEKLMKQIGVKNVKLSEYEMSIAAHLVDPLNMHVTWSDIAGLDDVITDLKDTVILPIKKKHLFENSRLLQPPKGVLLYGPPGCGKTLIAKATAKEAGCRFINLQPSTLTDKWYGESQKLAAAVFSLAIKLQPSIIFIDEIDSFLRNRSSSDHEATAMMKAQFMSLWDGLDTDHSCQVIVMGATNRPQDLDSAIMRRMPTRFHINQPALKQREAILKLILKNENVDRHVDLLEVAQETDGFSGSDLKEMCRDAALLCVREYVNSTSEESHDEDEIRPVQQQDLHRAIEKMKKSKDAAFQNVLTHVCLD

SEQ ID NO: 2 - protein, 361 aa, Mus musculus; identical to SEQ ID NO: 1